(19) United States
(12) Patent Application Publication
(10) Pub. No.: US 2026/0248805 A1
(43) Pub. Date: Aug. 27, 2026

(54) DIAMINOPHENOTHIAZINE FOR THE TREATMENT OF MICROVASCULAR BRAIN DISEASE

(71) Applicant: TauRx Therapeutics Management Ltd., Aberdeen (GB)

(72) Inventors: Claude Michel Wischik, Aberdeen, Aberdeenshire (GB); Shailendra Mohan Tripathi, Lucknow (IN); Alison Dorothy Murray, Portlethen, Aberdeenshire (GB); Roger Todd Staff, Aberdeen, Aberdeenshire (GB); Hellen Shiells, Aberdeen, Aberdeenshire (GB); Bjoern Olaf Schelter, Aberdeen, Aberdeenshire (GB)

(73) Assignee: TauRx Therapeutics Management Ltd., Aberdeen (GB)

(21) Appl. No.: 19/161,696

(22) PCT Filed: Feb. 28, 2024
(86) PCT No.: PCT/EP2024/055065

§ 371 Date: Sep. 2, 2025

(30) Foreign Application Priority Data
Mar. 3, 2023 (IN) 202311014304

(51) Int. Cl. A61K 31/5415; A61P 25/00; A61P 25/28

(52) U.S. Cl. CPC A61K 31/5415; A61P 25/00; A61P 25/28

(57) ABSTRACT

The invention provides a method of therapeutic or prophylactic treatment of microvascular brain disease in a subject, which method comprises orally administering to said subject reduced form of a methylthioninium (MT)-containing compound, wherein said administration provides a total daily dose of between 8 and 200 mg of MT to the subject per day, optionally split into 2 or more doses.

### TECHNICAL FIELD

The present invention relates generally to methods and materials for use in treating microvascular brain disease.

### BACKGROUND TO THE INVENTION

Microvascular brain disease arises from changes or loss of integrity in the small blood vessels in the brain, and is a common finding in older adults. There can be manifest as asymptomatic ischemic lesions, identified on computed tomography and magnetic resonance imaging (MRI) scans as white matter hyperintensities (WMH) or as lacunea (see Koton, Silvia, et al. “Microvascular brain disease progression and risk of stroke: the ARIC study.” Stroke 51.11 (2020): 3264-3270).

However, if left untreated microvascular disease can lead to cognitive decline, dementia, stroke, and other undesirable outcomes.

Treatment of microvascular brain disease is generally dependent on the suspected underlying cause, but may typically involve reducing or managing risk factors, such as high blood pressure, cholesterol level, diabetes and smoking. For example, subjects suffering from, or believed to be at high risk of, microvascular brain disease may be encouraged to adopting healthier lifestyles and may be prescribed medications to lower high blood pressure and control cholesterol and glucose levels.

Publication WO 2022/029606 reports that known pharmacological treatments for microvascular injuries include medications to control the narrowing of small blood vessels such as nitroglycerin, beta blockers, calcium channel blockers, statins, angiotensin-converting enzyme (ACE) inhibitors, angiotensin II receptor blockers (ARBs), Ranolazine (Ranexa) and aspirin. WO 2022/029606 reports that cannabinoid-based compositions may be used for preventing and/or treating damages to microvascular integrity, and thus treating conditions related injured microvasculature.

It can be seen that providing novel treatments for microvascular brain disease would provide useful contributions to the art.

### DISCLOSURE OF THE INVENTION

The present inventors have shown diaminophenothiazines are effective in reducing the rate of progression of white matter hyperintensities and microvascular pathology in subjects. This was demonstrated in a clinical trial over the course of a year of treatment.

The diaminophenothiazine compound used in the trial was a leucomethylthioninium (LMT) compound. Methylthioninium (MT) is a redox molecule and, depending on environmental conditions (e.g., pH, oxygen, reducing agents), exists in equilibrium between the reduced (LMT) and oxidized form (MT⁺).

There are reports that MT (in the form of Methylthioninium Chloride, MTC, also known as methylene blue) can delay progression of perfusion-diffusion mismatch to infarct in permanent ischemic stroke (see Rodriguez, Pavel, et al. “Methylene blue treatment delays progression of perfusion-diffusion mismatch to infarct in permanent ischemic stroke.” Brain research 1588 (2014): 144-149) and can be neuroprotective in ischemic stroke (see Shen, Qiang, et al. “Neuroprotective efficacy of methylene blue in ischemic stroke: an MRI study.” PloS one 8.11 (2013): e79833). Both these studies modelled ischaemic stroke modelled using occlusion of the middle cerebral artery and reported reduction in lesion volume following reperfusion. The precise mechanism for neuroprotection was unknown.

However neither study reported or suggested any effects for MTC in relation to microvascular pathology or white matter intensity progression in human subjects. An effect in this quite different pathology could not have been reasonably predicted from reported based on an occlusion event in a single large vessel.

The treatment of white matter hyperintensities and microvascular pathology demonstrated by the present inventors was in the context of a clinical trial for the treatment of mild to moderate Alzheimer's disease (AD).

MT acts as a tau aggregation inhibitor in vitro (Wischik C M, Edwards P C, Lai R Y K, Roth M, Harrington C R. Selective inhibition of Alzheimer disease-like tau aggregation by phenothiazines. Proc Natl Acad Sci USA 1996; 93: 11213-8; Harrington C R, Storey J M D, Clunas S, et al. Cellular models of aggregation-dependent template-directed proteolysis to characterize tau aggregation inhibitors for treatment of Alzheimer's disease. J Biol Chem 2015; 290: 10862-75.), dissolves PHFs from Alzheimer's disease brain tissue, (Wischik et al., 1996) and reduces tau pathology and associated behavioural deficits in transgenic mouse tau models at brain concentrations consistent with human oral dosing (Melis V, Magbagbeolu M, Rickard J E, et al. Effects of oxidized and reduced forms of methylthioninium in two transgenic mouse tauopathy models. Behav Pharmacol 2015; 26: 353-68; Baddeley T, C., McCaffrey J, Storey J M D, et al. Complex disposition of methylthioninium redox forms determines efficacy in tau aggregation inhibitor therapy for Alzheimer's disease. J Pharmacol Exptl Therapeutics 2015; 352: 110-8.). MT has also been shown to inhibit other disease-associated protein aggregation (see e.g. WO2007/110629 and references therein).

However MT or LMT has not previously been described for treatment of microvascular pathology. Indeed the fact that the load of microvascular pathology was a significant contributor to cognitive impairment in AD was quite unexpected, and even more surprising that it was seen in typical or “pure” AD and not in the mixed AD/vascular subtype.

Thus in one aspect there is disclosed a method of therapeutic or prophylactic treatment of microvascular brain disease in a subject, which method comprises orally administering to said subject a methylthioninium (MT)-containing compound, wherein the MT-containing compound is a salt of

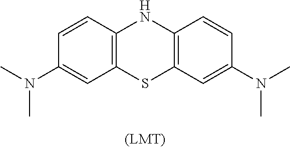

or a hydrate or solvate thereof.

“Microvascular brain disease” arises from changes or loss of integrity in the small blood vessels in the brain. Depending on the severity of these changes, they can cause a range of complications—from difficulty in focusing to a stroke. Thus the term embraces microvascular dysfunction in the brain leading to microvascular ischemic disease such as cerebral small vessel disease (CSVD), chronic microvascular ischemic (brain) disease; and small vessel ischemic disease. Cerebral microbleeds (MBs) are small chronic brain hemorrhages which are likely caused by structural abnormalities of the small vessels of the brain (Anand Viswanathan and Hugues Chabriat (2006) Cerebral microhemorrhage. Stroke. 37:550-555).

Such changes or loss of integrity could occur through injury but are believed to occur gradually even in normal aging subjects. Thus, microvascular ischemic disease occurs in older adults, affecting both males and females equally. It affects about 5% of people who are 50 years old. But it affects almost 100% of people older than 90. Microvascular ischemic disease is a very common condition in older people. According to estimates, it causes 45% of dementia and 25% of strokes. Transient ischaemic attacks (TIAs) or “mini strokes” are caused by a temporary disruption in the blood supply to part of the brain which may also arise from microvascular ischemic disease.

Suitable subjects and indications for treatment or prophylaxis according to the invention include the aforementioned, as well as those discussed in more detail hereinafter.

The methods of the invention have the purpose of reducing the rate of progression of microvascular pathology in the subject i.e. reduction of the increase in total lesion volume over time which would otherwise be seen in the same subject, or which is seen in a corresponding subject not receiving the treatment. The treatment timeframe will typically be at least 6 months, but may be longer, e.g. 1, 2, 3, 4 or 5 years or more. As shown in the Examples below microvascular pathology as measured by total volume of white matter hyperintensities progressed over the space of a year in subjects, but this progression could be inhibited by the use of an LMT compound.

Thus methods of the invention may be used with disease modifying effect on the microvascular pathology, for example for preventing and/or treating damages to microvascular integrity, and thus treating conditions related to injured microvasculature.

The methods may be used to reduce cognitive defects in the subject which would otherwise result from the progressive microvascular pathology.

The mechanism underlying the LMTM effect on microvascular pathology is unknown.

Without wishing to be bound by theory it is possible that it may operate by enhancement of mitochondrial metabolism. Reduction in treatment effect of LMTM by concomitant symptomatic treatments would be consistent with an effects on mitochondrial function (Atamna, H., Mackey, J. & Dhahbi, J. M. (2012) Mitochondrial pharmacology: electron transport chain bypass as strategies to treat mitochondrial dysfunction. Biofactors 38, 158-66; Riedel, G., Klein, J., Niewiadomska, G., Kondak, C., Schwab, K., Lauer, D., Magbagbeolu, M., Steczkowska, M., Zadrozny, M., Wydrych, M., Cranston, A., Melis, V., Santos, R. X., Theuring, F., Harrington, C. R. & Wischik, C. M. (2020) Mechanisms of anticholinesterase interference with tau aggregation inhibitor activity in a tau-transgenic mouse model. Current Alzheimer Research 17, 285-296).

Microvascular brain disease and microvascular pathology may be measured as total lesion volume (TLV) of hyperintensities seen on MRI. As explained herein, surprisingly the load of microvascular pathology was found to be a significant contributor to cognitive impairment in AD, but the load could be reduced by use of the compounds described herein.

In particular, treatment with LMT was shown to produce an exposure-dependent reduction in progression of microvascular pathology, thereby providing a novel approach to treatment of microvascular pathology in a subject.

In one embodiment the subject is a human.

In one embodiment the subject is between 40 and 89 years, for example at least 60 years, or about 70 years

In one embodiment the subject may be, or have been, diagnosed and/or selected as having said microvascular brain disease. For example based on imaging e.g. TLV as described above.

A further unexpected finding described herein is that hypertension was commoner in typical AD than the mixed subtype (AD and CVD) and that the association is driven by the hitherto unsuspected contribution of microvascular pathology to cognitive impairment in typical AD.

Thus in one embodiment the subject may be one diagnosed with pure AD (i.e. not mixed AD) and/or other indications related to microvascular pathology described herein.

Cerebrovascular disease (CVD) has risk factors including hypertension, diabetes, atherosclerosis, hypercholesterolemia, and dyslipidaemia.

As explained in the Examples below, although brain atrophy was the main driver of cognitive impairment in patients with AD, the microvascular pathology in the form of WMHs was significantly higher in patients with hypertension irrespective of subtype (p≤0.0001).

Thus treatment or prophylaxis in the present invention may be based on any of these risk factors, or based on other pre-disposing familial or genetic data.

WO2008/155533 describes the use of MT-compounds for the treatment of mild cognitive impairment. WO2021/001306 describes the use of MT-compounds for general cognitive function. Neither of these publications discuss a role for MT in microvascular brain disease, or any effects on microvascular pathology.

In some embodiments the subject may be one not suffering from AD of any type. For example, subjects in relation to the present invention will be those who do not suffer from, and have not been diagnosed with e.g. vascular dementia, senile dementia, age-associated memory impairment, Alzheimer's disease, Lewy body dementia, Parkinson's disease or mild cognitive impairment. Such subjects may thus be diagnosed not to suffer from these diseases. Diagnosis in this context can be according to the generally recognized criteria of The Diagnostic and Statistical Manual of Mental Disorders, 5th edition (DSM-5, American Psychiatric Association, 2013). Likewise, such subjects may not suffer from PTSD or a defect in mitochondrial energy metabolism.

For the purposes of the present invention, it is expected that a wide range of doses could be used (8 mg/day-200 mg/day).

Thus administration may provide a total daily dose of between 8 and 200 mg of MT to the subject per day, optionally split into 2 or more doses. However, it may be preferable to use smaller doses e.g. 8-60 mg/day e.g. 8 mg/day.

Based on the concentration dependence described hereinafter, the total daily MT dose may advantageously be between 12 and 60 mg. An example dosage is 16 to 32 mg. A further example dosage is 16 to 30 mg.

Thus total daily dose may be about 16, 17, 18, 19, 20, 21, 22, 23, 24, 25, 26, 27, 28, 29, 30, 31, or 32 mg

The present invention concerns administering MT in the reduced (LMT) form.

The total daily dose of the compound may be administered as a split dose twice a day or three times a day.

As explained below, when administering the MT dose split in a larger number of doses/day it may be desired to use a smaller total amount within the recited range, compared to a single daily dosing, or a smaller number of doses per day.

Current approved treatments for Alzheimer's disease include acetylcholinesterase inhibitors (AChEIs) and the N-methyl-D-aspartate receptor antagonist memantine.

The analysis in the Examples below shows that LMTM as monotherapy was associated with less increase in TLV than in subjects receiving LMTM in combination with these symptomatic treatments.

Thus in some embodiments the treatment will be a monotherapy, in the sense that it will exclude co-medication with AChEIs and memantine.

In some embodiments subjects are selected who have had not had recent prior treatment which AChEIs or memantine or other symptomatic treatments.

In other embodiments the treatment will be an add-on therapy, for example co-medication with AChEIs and\or memantine. Thus patients already receiving AChEIs and\or memantine may benefit from receiving these dosages of MT compound, while patients receiving these dosages of MT compound, may benefit from AChEIs and\or memantine.

The treatment may also be combined with other agents or approaches intended to treat the microvascular brain disease e.g. medications to lower high blood pressure and control cholesterol and glucose levels.

These aspects and embodiments will now be described in more detail: Methylthioninium moiety

| | |
|---|---|
| Structure | 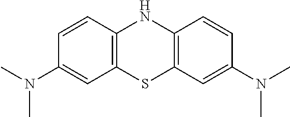 |
| IUPAC | N3,N3,N7,N7-tetramethyl-10H- |
| | phenothiazine-3,7-diamine |
| Composition | Formula Weight: 285.41(1) |
| | Exact Mass: 285.1299683(1) |
| | Formula: C₁₆H₁₉N₃S |
| | Composition: C 67.33% H 6.71% |
| | N 14.72% S 11.23% |
| Synonym | leucomethylthioninium (LMT) |

The MT-containing compounds used in the present invention contain an MT moiety as active ingredient in reduced form (termed “LMT”). The LMT moiety per se described above is not stable. It will therefore be administered as an LMT compound—for example an LMT salt.

LMT-containing compounds will generally be stabilised, for example by the presence of one or more protic acids e.g. two protic acids.

The MT content of such salts can be readily calculated by those skilled in the art based on the molecular weight of the compound, and the molecular weight of the MT moiety. Examples of such calculations are given herein.

WO2007/110627 disclosed certain 3,7-diamino-10H-phenothiazinium salts, effective as drugs or pro-drugs for the treatment of diseases including Alzheimer's disease and other diseases such as Frontotemporal dementia (FTD). These compounds are also in the “reduced” or “leuco” form when considered in respect of MTC. These leucomethylthioninium compounds were referred to as “LMTX” salts.

WO2012/107706 described other LMTX salts having superior properties to the LMTX salts listed above, including leuco-methylthioninium bis(hydromethanesulfonate), LMTM (USAN name hydromethylthionine mesylate, HMTM):

| | |
|---|---|
| 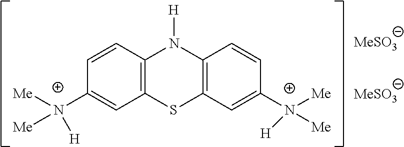 | N,N,N′,N′-tetramethyl-10H- phenothiazine-3,7- diaminium bis(hydromethanesulfonate). LMT.2MsOH / LMTM / HMTM |

Specifically LMTM retains TAI activity in vitro and in vivo (Harrington et al., 2015; Melis et al., 2015) has superior pharmaceutic properties in terms of solubility and pKa, and is not subject to the absorption limitations of the MT⁺ form (Baddeley et al., 2015).

WO2018/019823 describes novel regimens for treatment of neurodegenerative disorders utilising methylthioninium (MT)-containing compounds. Briefly, these regimens identified two key factors. The first was in relation to the dosage of MT compounds, and the second was their interaction with symptomatic treatments based on modulation of acetylcholinesterase levels.

In the analysis described in WO2018/019823, low doses of MT compounds (for example 4 mg b.i.d) showed therapeutic benefits when monotherapy was compared against add-on. The efficacy profiles were similar in mild and moderate subjects for most of the measured outcomes.

Furthermore, treatment benefit in AD (according to the trial criteria) was restricted to patients taking LMTM as monotherapy. By contrast, the decline seen at corresponding doses in patients taking LMTM in combination with AD-labelled treatments (acetylcholinesterase inhibitors [AChEIs] and\or memantine), who were the majority, was indistinguishable on all parameters from that seen in the control arm.

WO2018/019823 did not provide any teaching in relation to the effect of LMTM on microvascular brain disease, nor any teaching concerning interaction with AD-labelled treatments in treating microvascular brain disease.

WO2020/020751 describes devised a novel pharmacokinetic (PK) model for dosing MT compounds in patient populations. This versatile model was used to estimate Cmax of parent MT in patients who received LMTM in the two phase 3 trials of AD studies described in WO2018/019823 (Studies “005” and “015”, for treatment of mild, or mild to moderate, AD patients respectively, discussed in Example 1 below). Once the Cmax was estimated in each of the subjects, a distribution of Cmax estimates for each of the treated population could be derived. The novel analysis revealed that there exists a concentration response within the low dose treated population.

WO2020/020751 did not provide any teaching in relation to the effect of LMTM on microvascular brain disease, nor any teaching concerning an exposure dependence in such a treatment.

Preferred compounds for use in the present invention are “LMTX” compounds of the type described in WO2007/110627 or WO2012/107706.

Thus the compound may be selected from compounds of the following formula, or hydrates or solvates thereof:

| | |
|---|---|
| 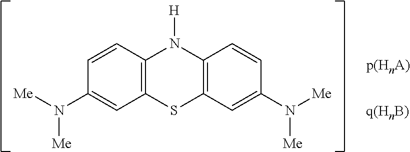 | Options: p = 1, 2 q = 0, 1 n = 1, 2 (p + q) × n = 2 |

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

| | |
|---|---|
| 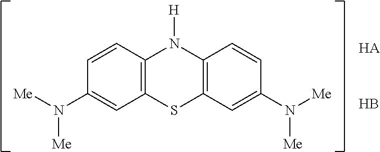 | when: p = 1 q = 1 n = 1 (1 + 1) × 1 = 2 |

However preferably the salt is not a mixed salt, and has the following formula:

| | |
|---|---|
| 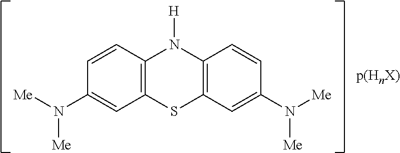 | when: p = 1, 2 n = 1, 2 p × n = 2 |

wherein each of HₙX is a protic acid, such as a di-protic acid or mono-protic acid.

In one embodiment the salt has the following formula, where H₂A is a di-protic acid:

| | |
|---|---|
| 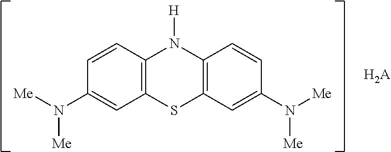 | when: p = 1 q = 0 n = 2 (1 + 0) × 2 = 2 |

Preferably the salt has the following formula which is a bis monoprotic acid:

| | |
|---|---|
| 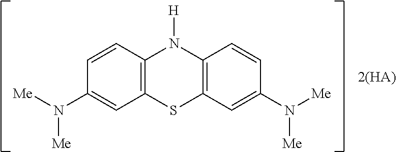 | when: p = 2 q = 0 n = 1 (2+ 0) × 1 = 2 |

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃COOH), methanesulfonic acid, 1,2-ethanedisulfonic acid, ethanesulfonic acid, naphthalenedisulfonic acid, p-toluenesulfonic acid,

Preferred acids are monoprotic acid, and the salt is a bis(monoprotic acid) salt.

A preferred MT compound is LMTM:

| | | | |
|---|---|---|---|
| 1 | 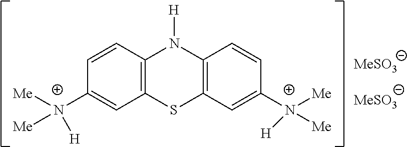 | LMT.2MsOH (LMTM) | 477.6 (1.67) |

### Weight Factors

The anhydrous salt has a molecular weight of around 477.6. Based on a molecular weight of 285.1 for the LMT core, the weight factor for using this MT compound in the invention is 1.67. By “weight factor” is meant the relative weight of the pure MT-containing compound vs. the weight of MT which it contains.

Other weight factors can be calculated for example MT compounds herein, and the corresponding dosage ranges can be calculated therefrom.

Other example LMTX compounds are as follows. Their molecular weight (anhydrous) and weight factor is also shown:

| | | | |
|---|---|---|---|
| 2 | 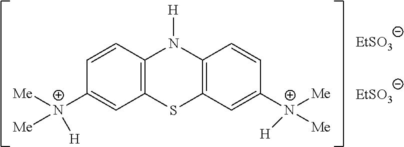 | LMT.2EsOH | 505.7 (1.77) |
| 3 | 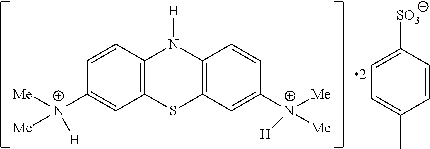 | LMT.2TsOH | 629.9 (2.20) |
| 4 | 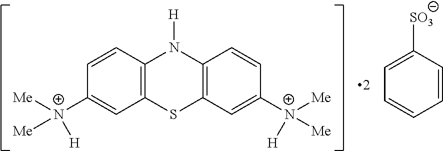 | LMT.2BSA | 601.8 (2.11) |
| 5 | 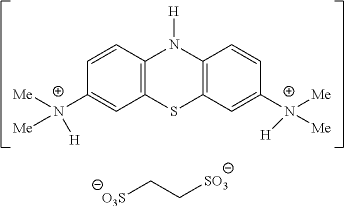 | LMT.EDSA | 475.6 (1.66) |
| 6 | 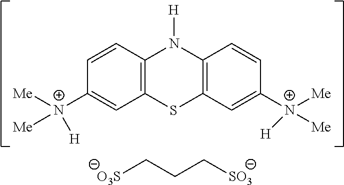 | LMT.PDSA | 489.6 (1.72) |
| 7 | 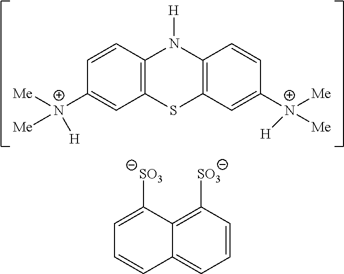 | LMT.NDSA | 573.7 (2.01) |
| 8 | 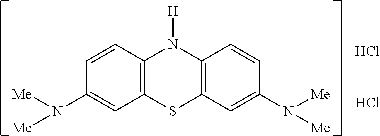 | LMT.2HCI | 358.33 (1.25) |

In some embodiments the LMT compound is not compound 8.

The dosages described herein with respect to MT thus apply mutatis mutandis for these MT-containing compounds, as adjusted for their molecular weight.

Methods for the chemical synthesis of the MT-containing compounds described herein are known in the art. For example:

Synthesis of compounds 1 to 7 can be performed according to the methods described in WO2012/107706, or methods analogous to those.

Synthesis of compound 8 can be performed according to the methods described in WO2007/110627, or a method analogous to those.

### Accumulation Factors

As will be appreciated by those skilled in the art, for a given daily dosage, more frequent dosing can lead to greater accumulation of a drug.

Therefore in certain embodiments of the claimed invention, the total daily dosed amount of MT compound may be relatively lower, when dosing more frequently (e.g. twice a day [bid] or three times a day [tid]), or higher when dosing once a day [qd].

### Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention can be much lower than was hitherto understood in the art.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

### Combination Treatments and Monotherapy

The term “treatment” includes “combination” treatments and therapies, in which two or more treatments or therapies for the same disorder are combined, for example, sequentially or simultaneously. These may be symptomatic or disease modifying co-treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention for the treatment of microvascular brain disease would be an agent which is an MT-containing compound at the specified dosage in combination with an agent which acts to lower blood pressure such as aspirin, candesartan and simvastatin (Smith and Markus, 2020, Stroke 51:38-46).

In other embodiments the treatment is a “monotherapy”, which is to say that the MT-containing compound is not used in combination (within the meaning discussed above) with another active agent for treating microvascular brain disease in the subject.

### Subjects, Patients and Patient Groups

The teachings of the invention may be applied to a subject/patient which is an animal, a mammal, a placental mammal, a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), a monotreme (e.g. platypus), an ape (e.g., gorilla, chimpanzee, orangutan, gibbon), or a human.

The patient may be an adult human, and the population-based dosages described herein are premised on that basis (typical weight 50 to 70 kg). If desired, corresponding dosages may be utilised for subjects falling outside of this range by using a subject weight factor whereby the subject weight is divided by 60 kg to provide the multiplicative factor for that individual subject.

### Labels, Instructions and Kits of Parts

Therapeutic or prophylactic compositions described herein may be provided in a labelled packet along with instructions for their use.

In one embodiment, the pack is a bottle, such as are well known in the pharmaceutical art. A typical bottle may be made from pharmacopoeial grade HDPE (high-density polyethylene) with a childproof, HDPE pushlock closure and contain silica gel desiccant, which is present in sachets or canisters. The bottle itself may comprise a label, and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding the disorder for which the medication is intended.

Said label or instructions may provide information regarding the maximum permitted daily dosage of the compositions as described herein—for example based on once daily, b.i.d., or t.i.d.

Said label or instructions may provide information regarding the suggested duration of treatment, as described herein.

### Methods of Treatment

Another aspect of the present invention, as explained above, pertains to a method of treatment comprising administering to a patient in need of treatment a prophylactically or therapeutically effective amount of a compound as described herein, preferably in the form of a pharmaceutical composition.

### Use in Methods of Therapy

Another aspect of the present invention pertains to a compound or composition as described herein, for use in a method of treatment (e.g., of a disease condition) of the human or animal body by therapy.

### Use in the Manufacture of Medicaments

Another aspect of the present invention pertains to use of an MT compound or composition as described herein, in the manufacture of a medicament for use in treatment (e.g., of a disease condition).

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a.” “an.” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES AND TABLES

FIG. 1: Examples of changes in FDG-PET patterns characteristic of metabolic activity in a patient with AD (A and C) and a mixed subtype (B and D) in axial, sagittal, and coronal planes. These images also confirm temporoparietal hypometabolism in AD. FDG uptake in mixed AD confirmed reduced uptake in vascular territories of the brain such as frontal and temporal lobes and the cerebellum.

FIG. 2: Different manifestations of cerebral small vessel disease including small subcortical infarct, white matter hyperintensity (WMH), lacune (usually with a hyperintense rim), perivascular space (mostly linear without a hyperintense rim) and cerebral microbleed (round or ovoid) (from Wardlaw, J. M, Smith, E. E., Biessels, G. J., Dichgans, M. (2013) Lancet Neurol, 12, 822-38).

FIG. 3: Illustration of WMHs from two subjects at three coronal planes. The upper panel shows occasional, small WMHs, the lower panel shows an example with numerous larger WMHs.

FIG. 4: Frequency of WMHs by various metrics (A, total Scheltens score; B, total lesion volume by LGA and C, WMH % by LGA) in an aging population (Aberdeen Birth Cohort (‘Children of the Nineteen Fifties’)).

FIG. 5: History of diabetes as a risk factor for WMH in an aging population. Figure shows raw WMH %.

FIG. 6: Exposure-dependent difference in rate of progression of microvascular pathology for LMTM as monotherapy (total lesion volume (TLV) measured in cm³), Modelled using mixed effect model repeated measured, change from baseline).

FIGS. 7A and 7B: LMTM as monotherapy at two different doses (4 and 100 mg b.i.d.) is associated with a lesser increase in TLV than in subjects receiving LMTM in combination with AChEI/Mem.

Table 1: Summary of baseline demographic and clinical features of the populations studied.

Table 2: Statistical analysis of baseline demographic and clinical characteristics with FDG-PET AD classification.

Table 3: Association between scan subtype and general vascular risk factors as recorded in patient's Case Report File.

Table 4: Frequency of cardiovascular risk factors.

Table 5: Association between microvascular pathology and sex in both AD subtypes.

Table 6: The association between ADAS-cog₁₁ score and microvascular pathology, brain atrophy, age, and sex in both AD subtypes.

Table 7: Comparison of microvascular pathology and brain fraction in the presence or absence of hypertension.

### EXAMPLE 1—BACKGROUND TO CLINICAL TRIAL

Dementia is a state of acquired intellectual deterioration which significantly interferes with the execution of personal, social or occupational functions, and hampers independent living of the individual, which is progressive and persists for at least 6 months. [1] Dementia is an over-arching term for a cluster of symptoms that can be caused by several diseases that affect the brain. Alzheimer's Disease (AD) is the most common brain pathology that causes dementia but frequently coexists with cerebrovascular disease (CVD). In the absence of definitive clinical diagnostic tests to differentiate dementia subtypes, brain imaging and fluid biomarkers are used to assist with aetiological diagnosis. A recent classification scheme based on the presence of positive markers for amyloid (Ap), tau and indicators of neurodegeneration has been proposed. [2,3] However, this suffers from the need to ascribe aetiological, diagnostic and ultimately therapeutic primacy to amyloid. Given the dissociation between amyloid pathology and cognitive decline, [4,5] the failure of numerous therapeutic approaches targeting the amyloid pathway [6] and the uncertain clinical benefits in clinical trials showing effects on amyloid load [7] it is open to question whether amyloid load is a valid surrogate marker that is predictive of clinical benefit. [8] If not, then the practical clinical relevance of defining AD in terms of amyloid pathology is also open to question.

A more important practical distinction in the aetiological subtyping of dementia is that between AD and CVD. These have specific characteristics and that can be identified by various imaging modalities and hence can contribute to differential diagnosis and help to better understand the pathological substrates of cognitive impairment during life. [9,10]. The last few decades have also witnessed the emergence of a growing literature supporting a possible role for vascular risk factors (VRFs) in the causation of AD. [11] The co-occurrence of AD and CVD pathology is more frequent than expected from age alone. [12] Reduction in cerebral perfusion is generally assumed to result from changes in the vascular system, leading to brain dysfunction and cognitive impairment. [13] It has been reported that AD and CVD have common risk factors including hypertension, diabetes, atherosclerosis, hypercholesterolemia, and apolipoprotein E4 genotype. [14] However, these relationships are not straightforward and depend on when and how risk factors and AD are defined, for example what stage of life hypertension operates and whether AD diagnosis is based on amyloid biomarkers. Among the common VRFs responsible for cognitive impairment, hypertension in midlife and diabetes in later life have relative risks of 1.6 and 1.5 respectively. [15] These associations have led to the emergence of the vascular hypothesis of AD. VRFs are proposed to have a causative role in developing the neuropathology of AD. [16] This has led to considerable interest in the potential for lifestyle and dietary interventions in the prevention of AD based on reduction of risk factors which overlap with VRFs. [15,17]

Structural and molecular imaging techniques have been used in the evaluation of patients with dementia in order to improve diagnostic accuracy. In comparative studies, 18-F-fluoro-deoxy-glucose-positron emission tomography (FDG-PET) appears to be superior to other diagnostic methods such as clinical diagnosis, magnetic resonance imaging (MRI), computed tomography (CT) and single photon emission computed tomography (SPECT) using technetium 99m hexamethylpropyleneamine oxime (HMPAO) tracer. [18-23] A valuable feature of FDG-PET is that it measures the functional consequences of pathology on brain metabolism. It is important to note AD and CVD commonly coexist and that patients with both forms of pathology have poorer cognition, worse prognosis and respond less well to candidate disease-modifying treatments in development. [24] Since AD and CVD are both independently associated with cognitive impairment, there are limitations in the extent to which the contribution of VRFs to the causation of AD pathology can be inferred based on clinical diagnostic criteria alone. If the combination of AD pathology and CVD lowers the threshold for detecting clinically significant cognitive impairment, risk factor attribution may be skewed towards ascribing to vascular factors a causal significance that they may not have.

A way of avoiding the confounding effect of biased case ascertainment is to use FDG-PET in patients meeting clinical diagnostic criteria for AD to distinguish those with only the typical temporoparietal metabolic deficits of AD from those with mixed AD/CVD changes. We reasoned that if typical vascular risk factors (hypertension, diabetes and dyslipidaemia) do indeed contribute to pure AD pathogenesis, this should be demonstrable in the subgroup of patients meeting both clinical and FDG-PET criteria for typical AD in the absence of evidence of mixed vascular pathology. If, on the other hand, VRFs contribute mainly to the mixed AD/CVD phenotype, then these factors should be over-represented in the mixed population. To examine this question, we have used baseline data from a large cohort of 794 patients available from two completed Phase 3 trials in mild to moderate AD [25,26] meeting National Institute of Aging and the Alzheimer's Association (NIA/AA) diagnostic criteria for probable AD [27] who had available FDG-PET scans at baseline and in whom VRFs had been documented in the trial case report forms.

### Material and Methods for Trial

### Patient Population

The design and results from two large Phase 3, double-blind, controlled, randomised clinical trials of hydromethylthionine in probable AD were described elsewhere. [25,26] Study TRx-237-015 (clintrials.gov NCT01689246), involved 890 mild/moderate AD patients and study TRx-237-005 (clintrials.gov NCT01689233), had 800 patients with mild AD meeting NIA/AA diagnostic criteria for probable AD. [27] Eligible patients were aged less than 90 years. Patients were excluded if they had: a recent history of poorly controlled hypertension, or clinically significant cardiovascular disease (such as hospitalization for acute coronary syndrome or symptoms consistent with angina pectoris), or a modified Hachinski ischemic score of >4. [26] Patients were also excluded if significant vascular pathology was seen on brain MRI, including large confluent white matter hyperintensities that would lead to a diagnosis other than probable AD. A detailed list of all inclusion and exclusion criteria for both trials is available in the supplementary materials. [25,26] FDG-PET was performed at baseline in only a subgroup of TRx-237-015 subjects (n=198) but was required for all participants in TRx-237-005 if the appropriate imaging facilities were available at the study site. [25,26] Of 1690 participants enrolled in both studies, 794 had available FDG-PET scans at baseline.

In both studies, medical history recorded in the case report form at baseline included, previous diagnosis of any vascular disorder and presence of hypertension, diabetes and dyslipidaemia. In addition, information regarding concomitant use of medications relevant to the treatment of potential vascular risk factors such as antihypertensives, insulin or other hypoglycaemic medications was also recorded.

### Classification of FDG-PET Images

FDG-PET images from 794 participants were classified visually into those with a typical AD imaging pattern and mixed AD/CVD pattern. Subjects with typical temporoparietal hypometabolism which was combined with deficits in one or more vascular territories were classified as having a mixed AD/CVD pattern, whereas those with only temporoparietal deficits were classified as having typical AD only. The classification was based on a visual review of scan images displayed in three planes using a standard colour scale representing FDG uptake using the PMOD Alzheimer's discrimination analysis tool (PALZ). Classification was undertaken independently by two observers, ADM and SMT. The inter-rater reliability, measured using Cohen's kappa, was 0.55 indicating an acceptable level of agreement. In cases of disagreement, images were reviewed jointly and discussed until a consensus was reached.

### MRI Brain Acquisition and Analysis

In this trial only GE, Siemens, or Philips (1.5-Tesla and 3.0-Tesla) machines were used to reduce the variability in the imaging data. The MRI sequences utilised in this protocol were based on the ADNI protocol. The recommended range of acquisition parameters for individual sequences for GE, Siemens, and Philips MRI machines were developed after technical evaluation forms from all sites. The following sequences were included: FLAIR: This sequence allows assessment of other clinically significant focal intracranial pathology and for detection and quantification of WMHs; Unenhanced T1-weighted, 3-dimensional sequence (e.g., MP RAGE or SPGR) were used for evaluation of the whole brain, ventricular and hippocampal volumes and WMH. MR images were also reviewed to ensure participants did not have a mass lesion or recent infarct.

Brain MRI derived WMHs were quantified using the Lesion Growth Algorithm (LGA) part of the Lesion Segmentation Toolbox (LST [28]) which is an automated method for extracting WMH and has been validated in various datasets. [29-31] This algorithm uses T1 and FLAIR MR images to classify voxels, with T1 images used to segment tissue classes white matter (WM), grey matter (GM), cerebrospinal fluid (CSF) and fluid attenuated inversion recovery (FLAIR) imaging used to determine areas of hyperintense signal (i.e., WMH). WMH data are provided as whole-brain total lesion volume (TLV), measured in cm³.

Brain MRI derived volumes were measured using Freesurfer to extract white matter, grey matter and cerebrospinal fluid volumes. The brain fraction (BF) was calculated as the ratio of the brain size (grey matter volume+white matter volume) to the total intracranial volume (TICV, grey matter volume+white matter volume+CSF).

### Statistics

Statistical analyses were performed using R version 3.5.1, employing paired samples t-tests to compare baseline characteristics in the AD imaging subtypes (Table 2). The chi-squared test was used to compare gender distribution for the AD subtypes (Tables 2 and 5). The association between AD subtypes and vascular risk factors (hypertension, diabetes, and dyslipidemia) was examined with binary logistic regression analysis (Table 3). In addition, regression analysis using generalized linear model was implemented to determine the association between AD subtypes and vascular risk factors, whilst controlling for age and sex (Table 4). Cognitive function at baseline was measured using the ADAS-cog₁₁ scale. The significance level was set at p<0.05.

Regression analysis using a linear model was used to determine the association between ADAS-cog₁₁ score and microvascular pathology, brain atrophy, age and sex. Relative contributions to the variance of the ADAS-cog11 score by covariates were assessed using partial R², and total model contribution to the variance was assessed using the coefficient of determination, R². This analysis was repeated for both AD subtypes (Table 6). Additionally, non-paired t-tests were used to compare microvascular pathology and brain fraction in the presence or absence of hypertension (Table 7).

### EXAMPLE 2—RESULTS OF TRIAL

### 2.1 Baseline Demographic

The Baseline demographic and clinical data for the 794 subjects are provided in Table 1. Mean age (±se) was 70.56±0.32 years, ranging from 40 to 89 years, with more females (55.2%). The overall frequency of vascular risk factors is also shown: about half had hypertension, about half had raised cholesterol and 14% were diabetic.

### 2.2 FDG-PET

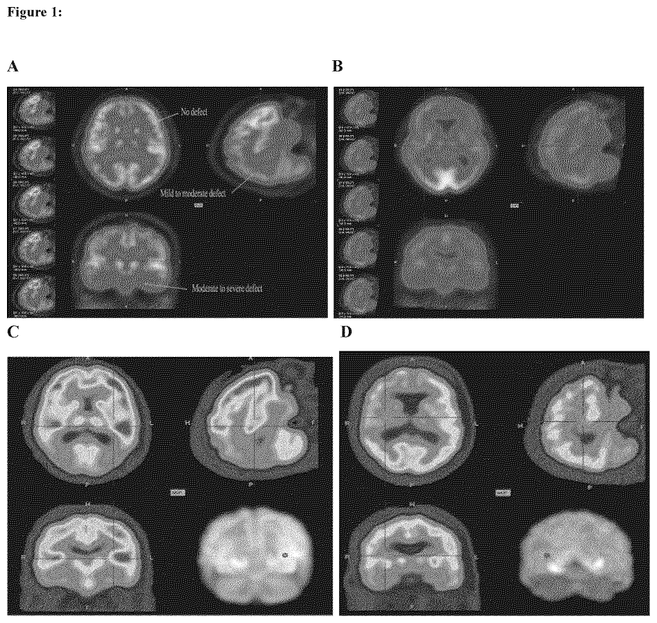

FDG-PET images were categorised into typical AD and mixed AD/CVD. Scans with a typical AD FDG-PET profile had decreased glucose uptake restricted to temporoparietal regions (FIG. 1A, C). Those with a mixed AD/CVD profile had reduced FDG uptake in a particular vascular territory, such as the middle cerebral artery and/or patchy uptake, in addition to typical temporoparietal hypometabolism (FIG. 1B, D). As such, the mixed AD/CVD category will have included those with large vessel disease.

Of 794 participants, 533 (67.1%, 47.1% male) were classified as having a typical AD scan pattern and 261 (32.8%, 41.0% male) were classified as having a mixed pattern (Table 2). Patients with a mixed AD/CVD scan profile were significantly younger (difference±se, p=0.002) and more cognitively impaired (difference±se, p<0.001) compared to those with a typical AD scan profile.

### 2.3 Vascular Risk Factors

We next analysed the association between scan subtype and general vascular risk factors as recorded in the patient case report form (cardiovascular, diabetes, dyslipidaemia). The distribution of the general vascular risk factors is shown in Table 3. Surprisingly, only cardiovascular factors differed in distribution between AD subtypes, and were more frequent in subjects with a typical AD scan (59%) than in those with a mixed scan (47%), a difference which was statistically significant (p=0.002). Although dyslipidaemia was also more frequent in those with a typical AD scan (typical 51%, mixed 45%), the difference was not significant (p=0.392). Likewise, the frequency of diabetes was comparable for the two groups (typical 14%, mixed 13%; p=0.567).

The conditions reported in the case report form classified as being specifically cardiovascular in nature are listed in Table 4. Hypertension was present in 52% (279/533) of the typical AD subgroup, whereas non-hypertension cardiac factors were present in only 10% (54/533) overall. However, their distribution is non-homogeneous (p<0.0001), with a higher frequency of non-hypertensive factors in hypertension patients. Hypertension was also more frequent in patients with a typical AD scan (52%) than in those with a mixed AD profile (42%), a difference which was statistically significant (p=0.0087). We compared the baseline blood pressure (sitting and standing) between subtypes to determine whether the risk factor could be attributed to presence of inadequately controlled blood pressure. There was no difference in blood pressure at baseline between the typical and mixed AD subtypes. Therefore, a history of adequately treated hypertension is the predominant cardiovascular risk factor associated with typical AD and is more frequent in this group than in patients with the mixed AD.

### 2.4 White Matter Hyperintensities and Brain Fraction

To better understand why hypertension is present more frequently in the typical AD subtype than in the mixed AD subtype, we used MRI scans available in the same patients to examine relationships between AD subtype and imaging metrics associated with cognitive impairment. These metrics were brain atrophy (as measured by brain fraction, BF) [32] and microvascular pathology (as measured by total lesion volume of white matter hyperintensities, TLV). [33,34] Although there were no overall differences between AD subtypes in terms of either brain atrophy or microvascular pathology, a sub-analysis showed that the lesion volume was significantly higher in typical than in mixed AD in patients who had been stratified as having a moderate impairment at baseline (MMSE 14-19, inclusive; 5.64±0.73 cm³ in typical AD and 3.54±0.56 cm³ in mixed AD, p=0.0268). This suggests that microvascular pathology may contribute to the severity of cognitive impairment in patients with typical AD. To investigate this further, we used a linear regression model to determine the relative contributions of microvascular pathology, brain atrophy, age and sex to dementia severity as measured by ADAS-cog₁₁ score at baseline in both the typical and mixed AD/CVD groups. Each variable was standardised (mean=0, standard deviation=1) to permit the linear coefficients in the analyses to be used directly to compare relative contributions of these factors. For the typical AD scan group, brain atrophy and microvascular pathology both made highly significant contributions to cognitive impairment, with the effect of brain atrophy being the larger of the two. Age and sex also contributed significantly to cognition in this group. This contrasts with the mixed AD group, where the main factor determining the level of cognitive impairment was brain atrophy, with a further significant contribution from age. Microvascular pathology and sex were not significant (Table 5). This can also be shown by examining the relative contributions to the variance in the ADAS-cog₁₁ score (Table 6). The overall model accounts for 16% of the variance for the typical AD subtype and 12% for the mixed AD subtype. Brain atrophy and microvascular pathology independently contribute 10% and 4% of the variance in the typical AD group, respectively. Brain atrophy is also the main contributor to variance (9%) in the mixed AD/CVD group. Only 1% of the variance is accounted for by microvascular pathology. Although these two forms of pathology contribute differently to cognitive impairment in the two AD subtypes, overall, there was a high and significant correlation between the two (r=−0.35, p=<0.0001).

Finally, we examined the effect of hypertension on the severity of microvascular pathology and whole-brain atrophy. As shown Table 7, the load of microvascular pathology is significantly higher in patients with hypertension irrespective of subtype. Similarly, the brain atrophy is marginally, but still significantly, greater in patients with hypertension. Therefore, hypertension contributes significantly to both microvascular pathology and brain atrophy.

### EXAMPLE 3—DISCUSSION OF FINDINGS IN CLINICAL TRIAL

The fundamental objective of this study was to examine the role of vascular risk factors in a large well characterized cohort of patients with mild to moderate AD meeting NIA/AA clinical diagnostic criteria for probable AD, who also had available FDG-PET and MRI scan data. The FDG-PET scans were used to classify patients according to whether they had typical temporoparietal deficits alone or in combination with defects in known vascular perfusion territories signifying a mixed AD/CVD phenotype. The expectation was that vascular risk factors (dyslipidemia, diabetes and hypertension) would have a greater prevalence in those with a mixed AD/CVD pattern. We found on the contrary that only the cardiovascular risk factors differed according to subtype and were more frequent in typical AD than in mixed AD/CVD subtypes. The predominant cardiovascular factor was found to be a history of clinically controlled hypertension. Further analysis revealed that patients with hypertension had a 34% higher load of white matter hyperintensities. In contrast, the brain fraction was reduced by less than 2%. Brain atrophy is the main contributor to cognitive impairment in both AD subtypes and in the mixed AD/CVD it was the only pathology making a significant contribution. Although it is known that a history of hypertension is a risk factor for microvascular pathology in the brain, [35] the main surprise from this study is that this contributes to cognitive impairment primarily in the typical AD subtype, and not at all in the mixed AD/CVD subtype. These unexpected findings help to understand better the role played by vascular risk factors in AD. They suggest that the contribution of vascular risk factors to the cognitive impairment in AD is mediated primarily via small vessel disease as measured by load of white matter hyperintensities (TLV). Where there are large vascular regions of reduced FDG uptake, as in the mixed group, the impact of the small vessel disease is not significant. Where there are no large vascular regions of reduced FDG uptake, the impact is significant. A history of hypertension is the predominant vascular risk factor in AD and this contributes selectively to the non-mixed subtype of AD.

Both brain atrophy and microvascular lesion load have a strong association with a history of hypertension. Indeed, a history of hypertension, even though currently well-controlled clinically, nevertheless has a highly deleterious impact on brain pathology and cognitive impairment, specifically in the typical AD subtype. The association between hypertension and having the typical AD profile on FDG-PET, and the lack of such an association for those with the mixed AD/CVD profile, are both surprising. An important motivation in undertaking the present analysis was to see whether the association between vascular risk factors and AD can be explained by the confounding effect of mixed AD/CVD pathology. Our results point to a much closer link between microvascular pathology and the pathology responsible for brain atrophy than we had initially hypothesised. Available reports linking AD with vascular risk factors tend to include hypertension, diabetes and dyslipidaemia as a group. [36] However, the association between hypertension and the typical AD subtype that we have identified does not apply to diabetes and dyslipidaemia.

We show that microvascular pathology as measured by total volume of hyperintensities seen by MRI provides an important link since this is well known to be associated both with hypertension and cognitive impairment. [37] Although brain atrophy is the main driver of cognitive impairment in patients with either AD subtype, microvascular lesion load is a significant contributor only in typical AD. This suggests that the association between vascular risk factors and AD which have been reported [38] may be determined by the presence of microvascular pathology. Comparing patients with and without hypertension, there is a 34% difference in the brain fraction, whereas the difference in microvascular lesion load is less than 2%. Therefore, it is unlikely that there is a simple volumetric relationship between these two parameters. An alternative explanation might be that the load of microvascular pathology is linked to a more general impairment in perfusion and tissue oxygenation which enhances neuronal vulnerability to either neurofibrillary or amyloid pathology. The load of tau aggregation pathology has been linked directly to brain atrophy. [39] The presence of hypertension has also been shown to increase susceptibility to AD [15,40-42] and has been linked to neurofibrillary tangle density. [43] Our data support a surprisingly high correlation of 0.35 between microvascular pathology and brain atrophy. Further studies that measure tau or amyloid pathology load more directly, and their relationship to microvascular pathology are required.

Only 67% of the population we have studied had the typical AD profile without deficits characteristic of cerebrovascular disease on FDG-PET scans, despite the use of MRI scans to exclude patients with marked CVD from inclusion in the trials. Moreover, the combination of clinical criteria and MRI scans failed to exclude the 33% of patients who did not have typical AD pattern on FDG-PET. This is comparable to the misdiagnosis rate when amyloid-PET criteria are used. [44] The main underlying cause of mixed AD/CVD pathology has been attributed to cerebral amyloid angiopathy (CAA). [24,45] If this true, then the recently proposed ATN classification system which requires evidence of amyloid pathology for an AD diagnosis may increase, rather than reduce, the confounding effect of CVD in diagnosis and in development of disease-modifying treatments. The contribution of amyloid to microvascular pathology is unknown, [46] but if increased in patients who are amyloid positive, may also increase the risk of amyloid-related imaging abnormalities in clinical trials targeting amyloid pathology. The diagnostic accuracy in distinguishing AD from healthy controls is comparable for FDG-PET and amyloid-PET at about 94%, although FDG-PET is superior in MCI. [47,48]

Typical and mixed AD differ in age and disease severity in our cohort. Mixed cases are younger and more severe. It has been reported previously that with advancing age there are increasing burdens of co-morbid AD pathology, while “pure AD” pathology decreases. [45,49] However, our results from a trial-derived population differ with those having a mixed AD/CVD phenotype being significantly younger compared to those with typical AD. The most likely explanation for this is that the co-existence of CVD with AD pathology reduced the threshold for a clinical diagnosis of AD, [50,51] and increased the likelihood of meeting the trial entry criteria. Nevertheless, the severity of cognitive impairment was found to be higher in patients with mixed pathology in comparison with those having the more typical AD pattern. This is in line with earlier findings that patients with “pure” AD pathology are older and have a less severe form of the disease in comparison to those with cerebrovascular disease. [52]

Dyslipidaemia has been considered a risk factor associated with AD. Increased cholesterol levels and its derivatives in the brain have been shown to induce neuronal apoptosis and oxidative stress, [53,54] potentially increasing the risk of developing dementia through neuronal loss and dysfunction. A study by Mielke et al. (2005) examined the total serum cholesterol levels in AD patients and found an association between high cholesterol levels and reduced risk of dementia in late life. [55] In contrast, follow-up studies have shown that high serum cholesterol levels in mid-life are associated with an increased risk for developing AD in late life. [56,57] Clinical trials have found no benefit in individuals with mild to moderate AD treated with statins (simvastatin), despite the significant lowering of cholesterol levels. [58] Whatever the role of hypercholesterolemia in the causation of AD, we report no difference in frequency of hypercholesterolemia between typical and mixed AD subtypes.

There has been substantial interest in a link between neurodegenerative disorders and metabolic dysfunction, including diabetes. [15,59] Diabetes has been reported to have the strongest link with AD amongst various vascular risk factors. [60] The Rotterdam study was among the first to show an elevated risk of dementia with type 2 diabetes. [61] Similarly, the Kungsholmen Project, a longitudinal population-based study, found that type 2 diabetes and pre-diabetes accelerated the progression from MCI to dementia by 3.18 years [62]. Other studies have also reported that diabetes shares similar mechanisms to AD including abnormalities in mitochondrial dysfunction, energy homeostasis, and neuroinflammation. [63] This has motivated anti-diabetic drug trials in AD. [64] However, none has shown promise so far. [65] Whatever the role of diabetes in the causation of AD, we report no difference in frequency between typical and mixed AD subtypes.

Although the present study is from a large and well-characterised population, it is a clinical trial population. As such, the extent to which the findings are applicable to a more general AD population is unknown. In the present study, we have used hypometabolism patterns from FDG-PET images to categorise patients according to typical AD and mixed AD/CVD subtypes. Therefore, the findings speak only to the question of whether vascular risk factors are equally represented in both subtypes. They leave open the possibility that the dyslipidaemia and diabetes could contribute equally to both subtypes. We show that patients with AD of either subtype are more severely impaired if they have hypertension. A further possibility explaining the relatively weaker role of hypertension in the mixed AD/CVD group could potentially be due to the exclusion criteria preventing enrolment of participants with significant vascular pathology or poorly controlled hypertension. Nevertheless, although there are fewer mixed cases (261) than typical AD cases (533) in this study, the numbers are sufficient to be informative. The proportion of cases without hypertension in the mixed AD/CVD group (58%) is significantly larger than in the typical AD group (48%).

In summary, we have used a large population of patients meeting clinical research criteria for a diagnosis of mild to moderate AD in an attempt to disentangle some of the confounding considerations in the discussion of the role of vascular risk factors in the causation of AD. A major difficulty in this discussion is the inclusion in populations diagnosed as having AD those who actually have mixed AD/CVD in many studies. We have used FDG-PET to try to distinguish typical AD from mixed. Contrary to the expectation, we show that hypertension is over-represented in the typical AD group compared with the mixed group. This is not the case for either dyslipidaemia or diabetes. This supports the idea that a history of hypertension (or the drugs used to treat hypertension) may have a role in typical AD that is not explained by the presence of mixed vascular and AD deficits that can be seen by FDG-PET.

Despite attempts to define “pure AD” solely based on evidence of amyloid pathology shown by biomarkers, there is no evidence that this has served to improve treatment efficacy in trials targeting amyloid pathology. [6] Indeed, the ATN system may have the perverse effect of confounding typical AD and mixed AD/CVD further, with important implications for the interpreting clinical trial results. Clinical classification of dementia remains controversial due to the complex nature of the diseases causing cognitive impairment and the difficulty in defining “pure AD” as a specific category using any of the available diagnostic criteria. [66] Recognising the distinction between AD and mixed AD/CVD remains difficult in clinical practice despite the availability of new biomarkers which aim to phenotype AD. [67] Mixed brain pathologies are the most common cause of cognitive impairment in the elderly and are the most common pathological finding in dementia patients. [45,68] This implies that whatever classification system one adopts, the reality of the underlying complexity is likely to persist in routine clinical practice and needs to be recognised in the developing the disease-modifying treatments for dementia.

### EXAMPLE 4—MICROVASCULAR BRAIN DISEASE IN THE AGING POPULATION

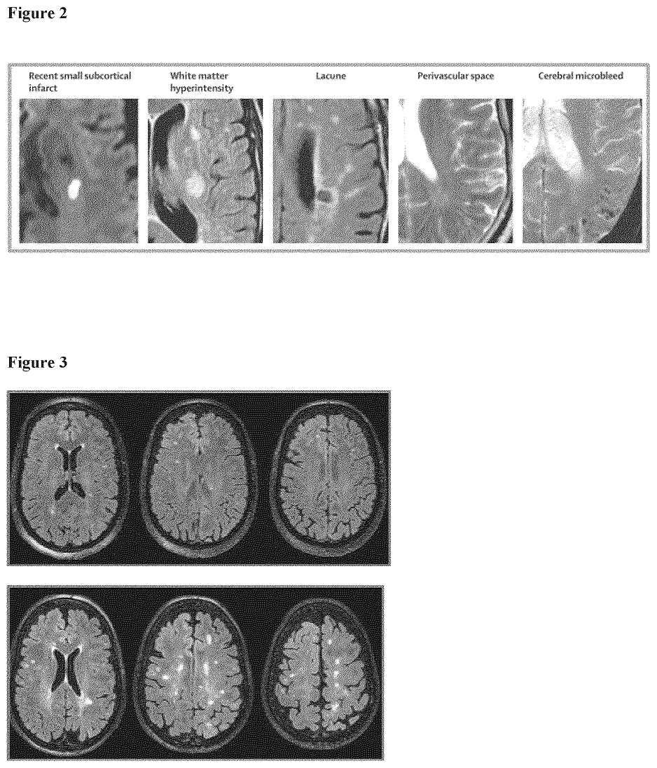

Microvascular brain disease (including cerebral small vessel disease) arises from changes or loss of integrity in the small blood vessels in the brain, and is a common finding in older adults. As shown in FIG. 2, manifestations of cerebral small vessel disease include subcortical infarct, white matter hyperintensity (WMH), lacune, perivascular space and cerebral microbleed.

FIG. 3 shows representative images of WMHs. It has been reported that WMHs predict an increased risk of stroke, gait disturbance, cognitive decline and vascular/mixed dementia. Risk factors include increasing age, hypertension, smoking and diabetes. The prevalence in the general population increases from around 11-21% (aged 64) to 94% (aged 82; see e.g. Debette, Stéphanie, and H. S. Markus. “The clinical importance of white matter hyperintensities on brain magnetic resonance imaging: systematic review and meta-analysis.” Bmj 341 (2010)).

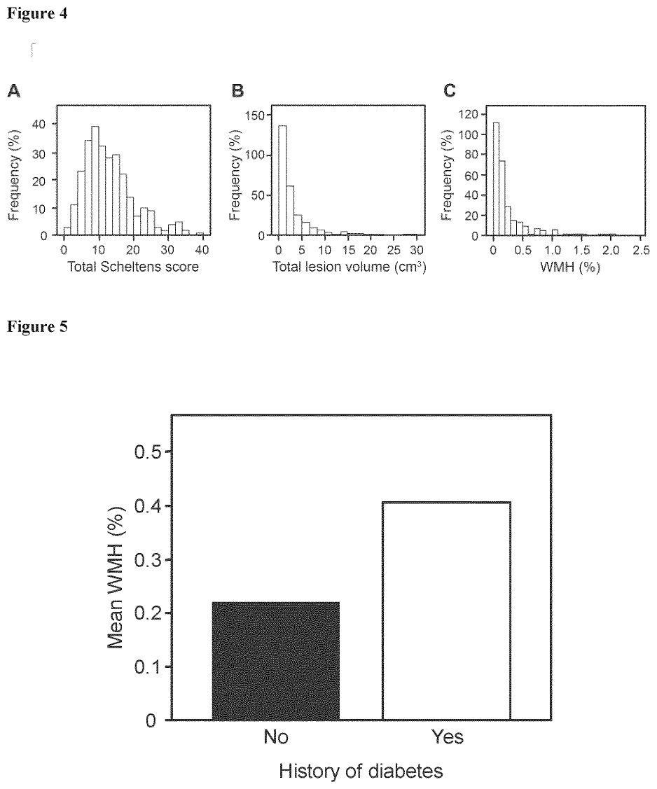

FIG. 4 shows the frequency of WMHs by various metrics (Scheltens, TLV, and %) in a population having a mean age of 62 (the Aberdeen Birth Cohort ‘Children of the Nineteen Fifties’ (‘ACONF’)—see Batty, G. David, et al. “The Aberdeen Children of the 1950s cohort study: background, methods and follow-up information on a new resource for the study of life course and intergenerational influences on health.” Paediatric and perinatal epidemiology 18.3 (2004): 221-239).

The prevalence data can be summarised as:

| | Mean | SD | Min | Max |
|---|---|---|---|---|
| Total Scheltens score | 12.70 | 7.28 | 1 | 39 |
| TLV (cm³) | 3.26 | 4.4 | 0 | 29.64 |
| WMH (%) | 0.24 | 0.32 | 0 | 2.08 |

(TLV—total lesion volume (LGA); WMH %—TLV as % of total intracranial volume)

In terms of risk factors for WMH, the data from ACONF shows that Increased WMH burden* correlates significantly with the following factors:
Increasing age (r=0.129, p=0.032)
Average systolic BP (r=0.163, p=0.007)
Diabetes (self-reported) (r=0.149, p=0.013)
(*WMH burden measured as WMH % (Log 10 transformed)
WMH %—total lesion volume as % total intracranial volume
Correlations—Pearson's r
Group differences—Independent-samples t-test

ACONF participants with diabetes had significantly greater WMH burden than those without: (t (275)=−2.49, p=0.013; see also FIG. 5).

Further analysis of the ACONF cohort (normal population) showed that increased WMH burden correlated significantly with poorer performance in the following cognitive tests*:
Digit symbol task (r=−0.133, p=0.028)
Mill Hill Vocabulary test (r=−0.141, p=0.019)
Matrix reasoning
   Total correct responses (r=−0.195, p=0.001)
   Total incorrect responses (r=0.193, p=0.001)
   Total time taken (r=−0.119, p=0.048)

*WMH burden measured as WMH % log₁₀ transformed; Correlations: Pearson's r

### EXAMPLE 5—TREATMENT OF MICROVASCULAR PATHOLOGY

Due to its prevalence in the aging population, and association with poorer cognitive outcomes (both in normal populations, as well as in typical AD), as well as being a risk factor for other undesirable outcomes, it can be seen that a treatment which reduces the rate of progression of microvascular pathology would provide a contribution to the art.

In the trial referred to in Examples 1-3, the present inventors have found that LMTM as monotherapy is associated with less increase in Total Lesion volume (TLV, measured in ml) than in subjects receiving LMTM in combination with symptomatic treatments for AD, with a statistically significant difference present in both high dose and low dose groups:

| Dosing | estimate | se | p-value |
|---|---|---|---|
| 4 mg b.i.d. | −0.34937 | 0.15227 | 0.021771 * |
| 100 mg b.i.d. | −0.38648 | 0.15127 | 0.010621 * (high dose) |

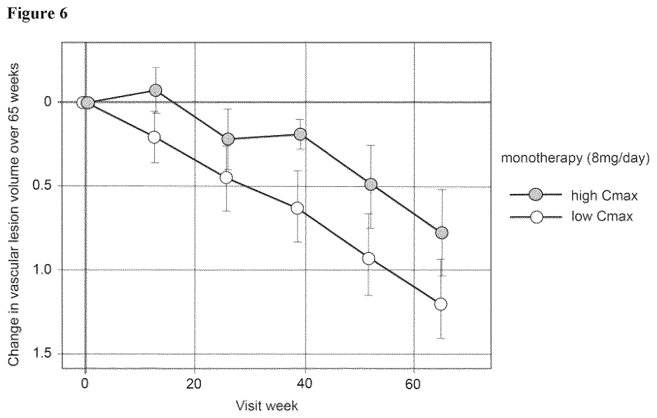

FIG. 6 shows the exposure-dependent difference in rate of progression of microvascular pathology for LMTM as a low dose monotherapy. Exposure dependence groups were defined in accordance with the methods described in more detail in WO2020/020751 discussed hereinbefore.

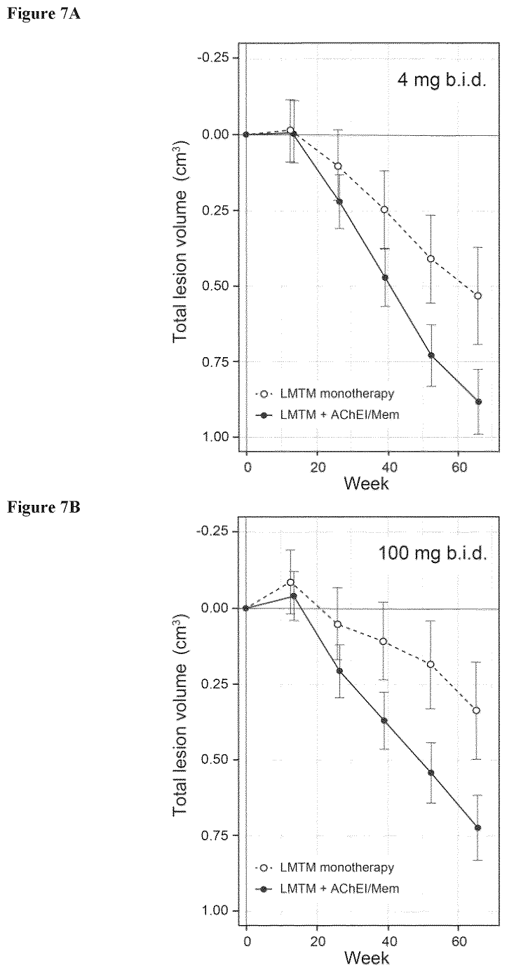

FIGS. 7A and B show that LMTM as monotherapy is associated with less increase in TLV than in subjects receiving LMTM in combination with AD symptomatic treatments.

A statistically significant difference present in both high dose (mean of high doses, 150-250 mg/day) and low dose groups:

| Dosing | estimate | se | p-value |
|---|---|---|---|
| 4 mg b.i.d. | −0.34937 | 0.15227 | 0.021771 * |
| 100 mg b.i.d. | −0.38648 | 0.15127 | 0.010621 * |

In conclusion, microvascular pathology measured as total lesion volume (TLV) of hyperintensities may be readily seen on MRI and increased microvascular pathology is associated with cognitive deficits measurable in normal aging subjects. Furthermore, the load of microvascular pathology appears to be a significant contributor to cognitive impairment in AD (surprisingly, this is seen in typical AD and not in the mixed AD/vascular subtype).

### EXAMPLE 6—DISCUSSION OF LITERATURE CONCERNING MICROVASCULAR PATHOLOGY IN NEURODEGENERATIVE DISEASE

Cerebral microvascular pathology precedes and accompanies age-related cognitive impairment and neurodegeneration (Bell and Zlokovic, 2009; Brown et al., 2009; Van Dijk et al., 2008). A gradual functional decline and a concomitant disintegrating morphology typically characterise the aging central nervous system (Farkas and Luiten, 2001). The physiological neuronal changes are also accompanied by a reduction in metabolic rates of oxygen and glucose, decreased cerebral blood flow as well as impaired structural integrity of cerebral blood vessels (Farkas and Luiten, 2001). These are all representative degenerative features of the vascular system of the aging brain. In AD, there is extensive literature linking changes in the vasculature to disease progression (Agrawal and Schneider, 2022; Farkas and Luiten, 2001; Steinman et al., 2021), with one study reporting up to one-third of AD patients with vascular pathology (Knopman et al., 2003), suggesting that there is a strong vascular component to cognitive impairment.

In addition to this, cerebrovascular lesions such as white matter hyperintensities (WMHs) are associated with an increased risk of clinical AD (Prins and Scheltens, 2015). WMHs are among the most prominent age-related changes observed on brain magnetic resonance imaging scans (Knopman et al., 2003). These WMH and lesions represent morphological changes to brain which are considered indicative of impaired brain function and, dependent on the affected brain area, can create specific symptoms (Jiménez-Balado et al., 2022). Increased WMH burden has also been associated with cognitive decline, increased risk of stroke and changes in gait.

The relationship between WMH burden and mild cognitive impairment (MCI) remains unclear, but some evidence suggests that WMH may increase the risk of MCI (Brickman et al., 2012; Tosto et al., 2015; Van Straaten et al., 2008). In a recent study of more than 350 elderly participants, the presence of WMHs was found to be associated with a substantial increase in risk of MCI, independent of grey matter volume, vascular risk factors, and vascular diseases (Boyle et al., 2016). Also, WMHs were associated with increased rates of cognitive decline, working memory, episodic memory, and semantic memory (Boyle et al., 2016).

In AD, WMHs are likely to add to underlying AD pathology (i.e., neurofibrillary tangles), therefore lowering the threshold for cognitive impairment (Corriveau et al., 2017; Silbert et al., 2009), and may contribute to AD pathophysiology (Garnier-Crussard et al., 2022). In the Rotterdam Study (de Leeuw et al., 2002), 87% of participants aged 60-70 years had subcortical WMHs, whereas 100% of participants aged 80-90 years had subcortical WMHs (de Leeuw et al., 2002). Furthermore, in AD, WMH volume appears to be larger than in cognitively unimpaired older adults (Maniega et al., 2015). In addition, an important recent study demonstrated that an increase in cortical tau load was associated with an increase in WMH severity (McAleese et al., 2015). Authors showed that in frontal, temporal and parietal regions, cortical tau burden predicted the severity of WMHs, independent of both cortical amyloid burden and small vessel disease severity (McAleese et al., 2015).

LMTM and related compounds may be used for preventing and/or treating damages to microvascular integrity, and thus treating conditions related injured microvasculature such as those described herein, in both the normal aging population, and subjects having AD. In either case it is preferable that it be used without ongoing prior treatment with AD-symptomatic drugs, although an effect in terms of reduced rate of progression (i.e. less increase in total lesion volume) may be seen regardless.

### REFERENCES FOR EXAMPLE 6

Agrawal, S., Schneider, J. A., 2022. Vascular pathology and pathogenesis of cognitive impairment and dementia in older adults. Cereb. Circ.—Cogn. Behav. 3, 100148. https://doi.org/10.1016/j.cccb.2022.100148
Bell, R. D., Zlokovic, B. V., 2009. Neurovascular mechanisms and blood-brain barrier disorder in Alzheimer's disease. Acta Neuropathol. 118, 103-113. https://doi.org/10.1007/s00401-009-0522-3
Boyle, P. A., Yu, L., Fleischman, D. A., Leurgans, S., Yang, J., Wilson, R. S., Schneider, J. A., Arvanitakis, Z., Arfanakis, K., Bennett, D. A., 2016. White matter hyperintensities, incident mild cognitive impairment, and cognitive decline in old age. Ann. Clin. Transl. Neurol. 3, 791-800. https://doi.org/10.1002/acn3.343
Brickman, A. M., Provenzano, F. A., Muraskin, J., Manly, J. J., Blum, S., Apa, Z., Stern, Y., Brown, T. R., Luchsinger, J. A., Mayeux, R., 2012. Regional white matter hyperintensity volume, not hippocampal atrophy, predicts incident Alzheimer disease in the community. Arch. Neurol. 69, 1621-1627. https://doi.org/10.1001/archneurol.2012.1527
Brown, W. R., Moody, D. M., Thore, C. R., Anstrom, J. A., Challa, V. R., 2009. Microvascular changes in the white mater in dementia. J. Neurol. Sci. 283, 28-31. https://doi.org/10.1016/j.jns.2009.02.328
Corriveau, R. A., Koroshetz, W. J., Gladman, J. T., Jeon, S., Babcock, D., Bennett, D. A., Carmichael, S. T., Dickinson, S. L. J., Dickson, D. W., Emr, M., Fillit, H., Greenberg, S. M., Hutton, M. L., Knopman, D. S., Manly, J. J., Marder, K. S., Moy, C. S., Phelps, C. H., Scott, P. A., Seeley, W. W., Sieber, B. A., Silverberg, N. B., Sutherland, M. L., Taylor, A., Torborg, C. L., Waddy, S. P., Gubitz, A. K., Holtzman, D. M., 2017. Alzheimer's disease-related dementias summit 2016: National research priorities. Neurology 89, 2381-2391. https://doi.org/10.1212/WNL.0000000000004717
de Leeuw, F.-E., de Groot, J. C., Oudkerk, M., Witteman, J. C. M., Hofman, A., van Gijn, J., Breteler, M. M. B., 2002. Hypertension and cerebral white matter lesions in a prospective cohort study. Brain 125, 765-772. https://doi.org/10.1093/brain/awf077
Farkas, E., Luiten, P. G. M., 2001. Cerebral microvascular pathology in aging and Alzheimer's disease. Prog. Neurobiol. 64, 575-611. https://doi.org/10.1016/S0301-0082(00)00068-X
Garnier-Crussard, A., Bougacha, S., Wirth, M., Dautricourt, S., Sherif, S., Landeau, B., Gonneaud, J., De Flores, R., de la Sayette, V., Vivien, D., Krolak-Salmon, P., Chételat, G., 2022. White matter hyperintensity topography in Alzheimer's disease and links to cognition. Alzheimers. Dement. 18, 422-433. https://doi.org/10.1002/alz.12410 Jiménez-Balado, J., Corlier, F., Habeck, C., Stern, Y., Eich, T., 2022. Effects of white matter hyperintensities distribution and clustering on late-life cognitive impairment. Sci. Rep. 12, 1955. https://doi.org/10.1038/s41598-022-06019-8
Knopman, D. S., Parisi, J. E., Boeve, B. F., Cha, R. H., Apaydin, H., Salviati, A., Edland, S. D., Rocca, W. A., 2003. Vascular dementia in a population-based autopsy study. Arch. Neurol. 60, 569-575. https://doi.org/10.1001/archneur.60.4.569
Maniega, S. M., Valdés Hernández, M. C., Clayden, J. D., Royle, N. A., Murray, C., Morris, Z., Aribisala, B. S., Gow, A. J., Starr, J. M., Bastin, M. E., Deary, I. J., Wardlaw, J. M., 2015. White matter hyperintensities and normal-appearing white matter integrity in the aging brain. Neurobiol. Aging 36, 909-918. https://doi.org/10.1016/j.neurobiolaging.2014.07.048
McAleese, K. E., Firbank, M., Dey, M., Colloby, S. J., Walker, L., Johnson, M., Beverley, J. R., Taylor, J. P., Thomas, A. J., O'Brien, J. T., Attems, J., 2015. Cortical tau load is associated with white matter hyperintensities. Acta Neuropathol. Commun. 3, 60. https://doi.org/10.1186/s40478-015-0240-0
Prins, N. D., Scheltens, P., 2015. White matter hyperintensities, cognitive impairment and dementia: An update. Nat. Rev. Neurol. 11, 157-165. https://doi.org/10.1038/nrneurol.2015.10
Silbert, L. C., Howieson, D. B., Dodge, H., Kaye, J. A., 2009. Cognitive impairment risk: White matter hyperintensity progression matters. Neurology 73, 120-125. https://doi.org/10.1212/WNL.0b013e3181ad53fd
Steinman, J., Sun, H. S., Feng, Z. P., 2021. Microvascular Alterations in Alzheimer's Disease. Front. Cell. Neurosci. 14, 618986. https://doi.org/10.3389/fncel.2020.618986
Tosto, G., Zimmerman, M. E., Hamilton, J. L., Carmichael, O. T., Brickman, A. M., 2015. The effect of white matter hyperintensities on neurodegeneration in mild cognitive impairment. Alzheimer's Dement. 11, 1510-1519. https://doi.org/10.1016/j.jalz.2015.05.014
Van Dijk, E. J., Prins, N. D., Vrooman, H. A., Hofman, A., Koudstaal, P. J., Breteler, M. M. B., 2008. Progression of cerebral small vessel disease in relation to risk factors and cognitive consequences: Rotterdam scan study. Stroke 39, 2712-2719. https://doi.org/10.1161/STROKEAHA.107.513176
Van Straaten, E. C. W., Harvey, D., Scheltens, P., Barkhof, F., Petersen, R. C., Thal, L. J., Jack, C. R., DeCarli, C., 2008. Periventricular white matter hyperintensities increase the likelihood of progression from amnestic mild cognitive impairment to dementia. J. Neurol. 255, 1302-1308. https://doi.org/10.1007/s00415-008-0874-y

### GENERAL REFERENCES

[1] Kurz A F, Lautenschlager N T. The concept of dementia: Retain, reframe, rename or replace?Int Psychogeriatrics 2010; 22:37-42. https://doi.org/10.1017/S1041610209991013.
[2] Jack C R, Bennett D A, Blennow K, Carrillo M C, Dunn B, Haeberlein S B, et al. NIA-AA Research Framework: Toward a biological definition of Alzheimer's disease. Alzheimer's Dement 2018; 14:535-62. https://doi.org/10.1016/j.jalz.2018.02.018.
[3] Dubois B, Villain N, Frisoni G B, Rabinovici G D, Sabbagh M, Cappa S, et al. Clinical diagnosis of Alzheimer's disease: recommendations of the International Working Group. Lancet Neurol 2021; 20:484-96. https://doi.org/10.1016/S1474-4422(21)00066-1.
[4] Weigand A J, Bangen K J, Thomas K R, Delano-Wood L, Gilbert P E, Brickman A M, et al. Is tau in the absence of amyloid on the Alzheimer's continuum?: A study of discordant PET positivity. Brain Commun 2020; 2:fcz046. https://doi.org/10.1093/braincomms/fcz046.
[5] Nelson P T, Alafuzoff I, Bigio E H, Bouras C, Braak H, Cairns N J, et al. Correlation of Alzheimer Disease Neuropathologic Changes With Cognitive Status: A Review of the Literature. J Neuropathol Exp Neurol 2012; 71:362-81. https://doi.org/10.1097/NEN.0b013e31825018f7.
[6] Reiss A B, Montufar N, DeLeon J, Pinkhasov A, Gomolin I H, Glass A D, et al. Alzheimer Disease Clinical Trials Targeting Amyloid: Lessons Learned From Success in Mice and Failure in Humans. Neurologist 2021; 26:52-61. https://doi.org/10.1097/NRL.0000000000000320.
[7] Huang L K, Chao S P, Hu C J. Clinical trials of new drugs for Alzheimer disease. J Biomed Sci 2020; 27:18. https://doi.org/10.1186/s12929-019-0609-7.
[8] Giannakopoulos P, Herrmann F R, Bussiere T, Bouras C, Kövari E, Perl D P, et al. Tangle and neuron numbers, but not amyloid load, predict cognitive status in Alzheimer's disease. Neurology 2003; 60:1495-500. https://doi.org/10.1212/01.WNL.0000063311.58879.01.
[9] Pasi M, Poggesi A, Pantoni L. The use of C T in dementia. Int Psychogeriatrics 2011; 23:S6-12. https://doi.org/10.1017/S1041610211000950.
[10] Patel K P, Wymer D T, Bhatia V K, Duara R, Rajadhyaksha C D. Multimodality imaging of dementia: Clinical importance and role of integrated anatomic and molecular imaging. Radiographics 2020; 40:200-22. https://doi.org/10.1148/rg.2020190070.
[11] Kivipelto M, Helkala E L, Laakso M P, Hanninen T, Hallikainen M, Alhainen K, et al. Midlife vascular risk factors and Alzheimer's disease in later life: Longitudinal, population based study. Br Med J 2001; 322:1447-51. https://doi.org/10.1136/bmj.322.7300.1447.
[12] Love S, Miners J S. Cerebrovascular disease in ageing and Alzheimer's disease. Acta Neuropathol 2016; 131:645-58. https://doi.org/10.1007/s00401-015-1522-0.
[13] Iadecola C. The Pathobiology of Vascular Dementia. Neuron 2013; 80:844-66. https://doi.org/10.1016/j.neuron.2013.10.008.
[14] Vijayan M, Reddy P H. Stroke, Vascular Dementia, and Alzheimer's Disease: Molecular Links. J Alzheimer's Dis 2016; 54:427-43. https://doi.org/10.3233/JAD-160527.
[15] Livingston G, Huntley J, Sommerlad A, Ames D, Ballard C, Banerjee S, et al. Dementia prevention, intervention, and care: 2020 report of the Lancet Commission. Lancet 2020; 396:413-46. https://doi.org/10.1016/S0140-6736(20)30367-6.
[16] de la Torre J. The Vascular Hypothesis of Alzheimer's Disease: A Key to Preclinical Prediction of Dementia Using Neuroimaging. J Alzheimers Dis 2018; 63:35-52. https://doi.org/10.3233/JAD-180004.
[17] Montero-Odasso M, Ismail Z, Livingston G. One third of dementia cases can be prevented within the next 25 years by tackling risk factors. The case “for” and “against.” Alzheimer's Res Ther 2020; 12:81. https://doi.org/10.1186/s13195-020-00646-x.
[18] Garibotto V, Herholz K, Boccardi M, Picco A, Varrone A, Nordberg A, et al. Clinical validity of brain fluorodeoxyglucose positron emission tomography as a biomarker for Alzheimer's disease in the context of a structured 5-phase development framework. Neurobiol Aging 2017; 52:183-95. https://doi.org/10.1016/j.neurobiolaging.2016.03.033.
[19] Heiss W D. The additional value of PET in the assessment of cerebral small vessel disease. J Nucl Med 2018; 59:1660-4. https://doi.org/10.2967/jnumed.118.214270.
[20] Kerrouche N, Herholz K, Mielke R, Holthoff V, Baron J C. 18FDG PET in vascular dementia: Differentiation from Alzheimer's disease using voxel-based multivariate analysis. J Cereb Blood Flow Metab 2006; 26:1213-21. https://doi.org/10.1038/sj.jcbfm.9600296.
[21] Mielke R, Pietrzyk U, Jacobs A, Fink G R, Ichimiya A, Kessler J, et al. HMPAO SPET and FDG PET in Alzheimer's disease and vascular dementia: comparison of perfusion and metabolic pattern. Eur J Nucl Med 1994; 21:1052-60. https://doi.org/10.1007/BF00181059.
[22] Marcus C, Mena E, Subramaniam R M. Brain PET in the diagnosis of Alzheimer's disease. Clin Nucl Med 2014; 39:e413-26. https://doi.org/10.1097/RLU.0000000000000547.
[23] Bloudek L M, Spackman D E, Blankenburg M, Sullivan S D. Review and meta-analysis of biomarkers and diagnostic imaging in Alzheimer's disease. J Alzheimer's Dis 2011; 26:627-45. https://doi.org/10.3233/JAD-2011-110458.
[24] Kapasi A, DeCarli C, Schneider J A. Impact of multiple pathologies on the threshold for clinically overt dementia. Acta Neuropathol 2017; 134:171-86. https://doi.org/10.1007/s00401-017-1717-7.
[25] Gauthier S, Feldman H H, Schneider L S, Wilcock G K, Frisoni G B, Hardlund J H, et al. Efficacy and safety of tau-aggregation inhibitor therapy in patients with mild or moderate Alzheimer's disease: a randomised, controlled, double-blind, parallel-arm, phase 3 trial. Lancet 2016; 388:2873-84. https://doi.org/10.1016/S0140-6736(16)31275-2.
[26] Wilcock G K, Gauthier S, Frisoni G B, Jia J, Hardlund J H, Moebius H J, et al. Potential of low dose leuco-methylthioninium bis(Hydromethanesulphonate) (LMTM) monotherapy for treatment of mild Alzheimer's disease: cohort analysis as modified primary Outcome in a Phase III clinical trial. J Alzheimer's Dis 2018; 61:435-57. https://doi.org/10.3233/JAD-170560.
[27] McKhann G M, Knopman D S, Chertkow H, Hyman B T, Jack C R, Kawas C H, et al. The diagnosis of dementia due to Alzheimer's disease: Recommendations from the National Institute on Aging-Alzheimer's Association workgroups on diagnostic guidelines for Alzheimer's disease. Alzheimer's Dement 2011; 7:263-9. https://doi.org/10.1016/j.jalz.2011.03.005.
[28] Schmidt P, Gaser C, Arsic M, Buck D, Förschler A, Berthele A, et al. An automated tool for detection of FLAIR-hyperintense white-matter lesions in Multiple Sclerosis. Neuroimage 2012; 59:3774-83. https://doi.org/10.1016/j.neuroimage.2011.11.032.
[29] de Sitter A, Steenwijk M D, Ruet A, Versteeg A, Liu Y, van Schijndel R A, et al. Performance of five research-domain automated W M lesion segmentation methods in a multi-center M S study. Neuroimage 2017; 163:106-14. https://doi.org/10.1016/j.neuroimage.2017.09.011.
[30] Maldjian J A, Whitlow C T, Saha B N, Kota G, Vandergriff C, Davenport E M, et al. Automated white matter total lesion volume segmentation in diabetes. Am J Neuroradiol 2013; 34:2265-70. https://doi.org/10.3174/ajnr.A3590.
[31] Waymont J M J, Petsa C, McNeil C J, Murray A D, Waiter G D. Validation and comparison of two automated methods for quantifying brain white matter hyperintensities of presumed vascular origin. J Int Med Res 2019; 48:300060519880053. https://doi.org/10.1177/0300060519880053.
[32] Sapkota S, Ramirez J, Yhap V, Masellis M, Black S E. Brain atrophy trajectories predict differential functional performance in Alzheimer's disease: Moderations with apolipoprotein E and sex. Alzheimer's Dement Diagnosis, Assess Dis Monit 2021; 13:e12244. https://doi.org/10.1002/dad2.12244.
[33] Prins N D, Scheltens P. White matter hyperintensities, cognitive impairment and dementia: An update. Nat Rev Neurol 2015; 11:157-65. https://doi.org/10.1038/nrneurol.2015.10.
[34] Hu H Y, Ou Y N, Shen X N, Qu Y, Ma Y H, Wang Z T, et al. White matter hyperintensities and risks of cognitive impairment and dementia: A systematic review and meta-analysis of 36 prospective studies. Neurosci Biobehav Rev 2021; 120:16-27. https://doi.org/10.1016/j.neubiorev.2020.11.007.
[35] Sierra C. Essential Hypertension, Cerebral White Matter Pathology and Ischemic Stroke. Curr Med Chem 2014; 21:2156-64. https://doi.org/10.2174/0929867321666131227155140.
[36] Chui H C, Zheng L, Reed B R, Vinters H V., MacK W J. Vascular risk factors and Alzheimer's disease: Are these risk factors for plaques and tangles or for concomitant vascular pathology that increases the likelihood of dementia? An evidence-based review. Alzheimer's Res Ther 2012; 4:1. https://doi.org/10.1186/alzrt98.
[37] Ungvari Z, Toth P, Tarantini S, Prodan Cl, Sorond F, Merkely B, et al. Hypertension-induced cognitive impairment: from pathophysiology to public health. Nat Rev Nephrol 2021; 17:639-54. https://doi.org/10.1038/s41581-021-00430-6.
[38] Steinman J, Sun H S, Feng Z P. Microvascular Alterations in Alzheimer's Disease. Front Cell Neurosci 2021; 14:618986. https://doi.org/10.3389/fncel.2020.618986.
[39] Schafer A, Chaggar P, Thompson T B, Goriely A, Kuhl E. Predicting brain atrophy from tau pathology: a summary of clinical findings and their translation into personalized models. Brain Multiphysics 2021; 2:100039. https://doi.org/10.1016/j.brain.2021.100039.
[40] Walker K A, Power M C, Gottesman R F. Defining the Relationship Between Hypertension, Cognitive Decline, and Dementia: a Review. Curr Hypertens Rep 2017; 19:24. https://doi.org/10.1007/s11906-017-0724-3.
[41] Dickstein D L, Walsh J, Brautigam H, Stockton S D, Gandy S, Hof P R. Role of vascular risk factors and vascular dysfunction in Alzheimer's disease. Mt Sinai J Med 2010; 77:82-102. https://doi.org/10.1002/msj.20155.
[42] Gottesman R F, Schneider A L C, Albert M, Alonso A, Bandeen-Roche K, Coker L, et al. Midlife hypertension and 20-year cognitive change: The atherosclerosis risk in communities neurocognitive study. JAMA Neurol 2014; 71:1218-27. https://doi.org/10.1001/jamaneurol.2014.1646.
[43] Sparks D L, Scheff S W, Liu H, Landers T M, Coyne C M, Hunsaker J C. Increased incidence of neurofibrillary tangles (NFT) in non-demented individuals with hypertension. J Neurol Sci 1995; 131:162-9. https://doi.org/10.1016/0022-510X(95)00105-B.
[44] Mosconi L, McHugh P F. FDG- and amyloid-PET in Alzheimer's disease: Is the whole greater than the sum of the parts?Q J Nucl Med Mol Imaging 2011; 55:250-64.
[45] Jellinger K A, Attems J. Prevalence of dementia disorders in the oldest-old: An autopsy study. Acta Neuropathol 2010; 119:421-33. https://doi.org/10.1007/s00401-010-0654-5.
[46] Thal D R, Attems J, Ewers M. Spreading of amyloid, tau, and microvascular pathology in alzheimer's disease: Findings from neuropathological and neuroimaging studies. J Alzheimer's Dis 2014; 42:S421-9. https://doi.org/10.3233/JAD-141461.
[47] Ottoy J, Niemantsverdriet E, Verhaeghe J, De Roeck E, Struyfs H, Somers C, et al. Association of short-term cognitive decline and MCI-to-AD dementia conversion with CSF, MRI, amyloid- and 18 F-FDG-PET imaging. NeuroImage Clin 2019; 22:101771. https://doi.org/10.1016/j.nicl.2019.101771.
[48] Rice L, Bisdas S. The diagnostic value of FDG and amyloid PET in Alzheimer's disease-A systematic review. Eur J Radiol 2017; 94:16-24. https://doi.org/10.1016/j.ejrad.2017.07.014.
[49] Jellinger K A, Attems J. Challenges of multimorbidity of the aging brain: a critical update. J Neural Transm 2015; 122:505-21. https://doi.org/10.1007/s00702-014-1288-x.
[50] Attems J, Jellinger K A. The overlap between vascular disease and Alzheimer's disease—lessons from pathology. BMC Med 2014; 12:206. https://doi.org/10.1186/s12916-014-0206-2.
[51] Esiri M M, Joachim C, Sloan C, Christie S, Agacinski G, Bridges L R, et al. Cerebral subcortical small vessel disease in subjects with pathologically confirmed alzheimer disease: A clinicopathologic study in the oxford project to investigate memory and ageing (OPTIMA). Alzheimer Dis Assoc Disord 2014; 28:30-5. https://doi.org/10.1097/WAD.0b013e31829b72f1.
[52] Zekry D, Duyckaerts C, Moulias R, Belmin J, Geoffre C, Herrmann F, et al. Degenerative and vascular lesions of the brain have synergistic effects in dementia of the elderly. Acta Neuropathol 2002; 103:481-7. https://doi.org/10.1007/s00401-001-0493-5.
[53] Marwarha G, Ghribi O. Does the oxysterol 27-hydroxycholesterol underlie Alzheimer's disease-Parkinson's disease overlap? Exp Gerontol 2015; 68:13-8. https://doi.org/10.1016/j.exger.2014.09.013.
[54] Zhao X S, Wu Q, Peng J, Pan L H, Ren Z, Liu H T, et al. Hyperlipidemia-induced apoptosis of hippocampal neurons in apoE(−/−) mice may be associated with increased PCSK9 expression. Mol Med Rep 2017; 15:712-8. https://doi.org/10.3892/mmr.2016.6055.
[55] Mielke M M, Zandi P P, Sjögren M, Gustafson D, Ostling S, Steen B, et al. High total cholesterol levels in late life associated with a reduced risk of dementia. Neurology 2005; 64:1689-95. https://doi.org/10.1212/01.WNL.0000161870.78572.A5.
[56] Whitmer R A, Sidney S, Selby J, Claiborne Johnston S, Yaffe K. Midlife cardiovascular risk factors and risk of dementia in late life. Neurology 2005; 64:277-81. https://doi.org/10.1212/01.WNL.0000149519.47454.F2.
[57] Solomon A, Kivipelto M, Wolozin B, Zhou J, Whitmer R A. Midlife serum cholesterol and increased risk of Alzheimer's and vascular dementia three decades later. Dement Geriatr Cogn Disord 2009; 28:75-80. https://doi.org/10.1159/000231980.
[58] Sano M, Bell K L, Galasko D, Galvin J E, Thomas R G, Van Dyck C H, et al. A randomized, double-blind, placebo-controlled trial of simvastatin to treat Alzheimer disease. Neurology 2011; 77:556-63. https://doi.org/10.1212/WNL.0b013e318228bf11.
[59] Baglietto-Vargas D, Shi J, Yaeger D M, Ager R, LaFerla F M. Diabetes and Alzheimer's disease crosstalk. Neurosci Biobehav Rev 2016; 64:272-87. https://doi.org/10.1016/j.neubiorev.2016.03.005.
[60] Luchsinger J A, Reitz C, Honig L S, Tang M X, Shea S, Mayeux R. Aggregation of vascular risk factors and risk of incident Alzheimer disease. Neurology 2005; 65:545-51. https://doi.org/10.1212/01.wnl.0000172914.08967.dc.
[61] Ott A, Stolk R P, Hofman A, Van Harskamp F, Grobbee D E, Breteler M M B. Association of diabetes mellitus and dementia: The Rotterdam Study. Diabetologia 1996; 39:1392-7. https://doi.org/10.1007/s001250050588.
[62] Xu W, Caracciolo B, Wang H X, Winblad B, BAckman L, Qiu C, et al. Accelerated progression from mild cognitive impairment to dementia in people with diabetes. Diabetes 2010; 59:2928-35. https://doi.org/10.2337/db10-0539.
[63] Chatterjee S, Mudher A. Alzheimer's disease and type 2 diabetes: A critical assessment of the shared pathological traits. Front Neurosci 2018; 12:383. https://doi.org/10.3389/fnins.2018.00383.
[64] Femminella G D, Bencivenga L, Petraglia L, Visaggi L, Gioia L, Grieco F V, et al. Antidiabetic Drugs in Alzheimer's Disease: Mechanisms of Action and Future Perspectives. J Diabetes Res 2017; 2017:7420796. https://doi.org/10.1155/2017/7420796.
[65] Munõz-Jiménez M, Zaarkti A, Garciá-Arnés J A, Garciá-Casares N. Antidiabetic Drugs in Alzheimer's Disease and Mild Cognitive Impairment: A Systematic Review. Dement Geriatr Cogn Disord 2021; 49:423-34. https://doi.org/10.1159/000510677.
[66] McKeith I G, Galasko D, Kosaka K, Perry E K, Dickson D W, Hansen L A, et al. Consensus guidelines for the clinical and pathologic diagnosis of dementia with Lewy bodies (DLB): Report of the consortium on DLB international workshop. Neurology 1996; 47:1113-24. https://doi.org/10.1212/WNL.47.5.1113.
[67] Zetterberg H, Blennow K. Moving fluid biomarkers for Alzheimer's disease from research tools to routine clinical diagnostics. Mol Neurodegener 2021; 16:10. https://doi.org/10.1186/s13024-021-00430-x.
[68] Matthews F E, Brayne C, Lowe J, McKeith I, Wharton S B, Ince P. Epidemiological pathology of dementia: Attributable-risks at death in the medical research council cognitive function and ageing study. PLoS Med 2009; 6:e1000180. https://doi.org/10.1371/journal.pmed.1000180.

TABLE 1
Summary of baseline demographic and clinical
features of the populations studied.

| | Mean or Frequency |
|---|---|
| Demographic and clinical | |
| Number | 794 |
| Age, yr (mean ± se) | 70.56 ± 0.32 |
| Range | 40-89 |
| MMSE score (mean ± se) | 22.15 ± 0.10 |
| Range | 14-26 |
| Sex n (%) | |
| Male | 356 (44.8) |
| Female | 438 (55.2) |
| Vascular risk factors | |
| Hypertension n (%) | |
| Present | 389 (49.0) |
| Absent | 405 (51.0) |
| Diabetes n (%) | |
| Present | 110 (13.9) |
| Absent | 684 (86.1) |
| Dyslipidaemia n (%) | |
| Present | 392 (49.4) |
| Absent | 402 (50.6) |

TABLE 2
Statistical analysis of baseline demographic and clinical
characteristics with FDG-PET AD classification.

| Parameters | Typical AD | Mixed AD | p-Value |
|---|---|---|---|
| Number, n (%) | 533 (67.1) | 261 (32.8) | |
| Mean age, yr (±se) | 71.23 (0.39) | 69.17 (0.55) | 0.002 |
| ADAS-cog (±se) | 17.89 (0.340) | 21.69 (0.556) | <0.001 |
| Sex, n (%) | | | |
| Male | 249 (46.71) | 107 (40.99) | 0.148 |
| Female | 284 (53.28) | 154 (59.01) | |

TABLE 3
Association between scan subtype and general vascular
risk factors as recorded in patient's Case Report File.

| Risk factor | Typical AD | Mixed AD | Effect (SE) | p-Value |
|---|---|---|---|---|
| Cardiovascular, | | | | |
| n (%) | | | | |
| Present | 316 (59) | 121 (47) | 0.509 (0.161) | 0.002 |
| Absent | 217 (41) | 138 (51) | | |
| Diabetes, n (%) | | | | |
| Present | 75 (14.07) | 35 (13.40) | −0.169 (0.233) | 0.467 |
| Absent | 458 (84.99) | 226 (86.59) | | |
| Dyslipidaemia, | | | | |
| n (%) | | | | |
| Present | 274 (51.40) | 118 (45.21) | 0.138 (0.161) | 0.392 |
| Absent | 259 (48.59) | 143 (54.78) | | |

TABLE 4
Frequency of cardiovascular risk factors.

| Cardiovascular risk factor | N present | N not present |
|---|---|---|
| Aortic necrosis and vascular insufficiency | 12 | 958 |
| Aortic valvular disorders | 9 | 961 |
| Arterial therapeutic procedures (excl aortic) | 14 | 956 |
| Cardiac conduction disorders | 26 | 944 |
| Ischaemic coronary artery disorders | 15 | 955 |
| Mitral valvular disorders | 25 | 945 |
| Myocardial disorders NEC | 12 | 958 |
| Vascular hypertensive disorders NEC | 472 | 498 |
| Non-hypertension cardiac risk factors | 54 | 479 |

TABLE 5
Association between microvascular pathology
and sex in both AD subtypes.

| | Coefficient ± SE | p-value |
|---|---|---|
| Typical AD | | |
| Microvascular pathology (TLV) | 1.33 ± 0.37 | 0.0004 |
| Brain fraction (BF) | −2.76 ± 0.38 | <0.0001 |
| Age | −0.86 ± 0.37 | 0.0210 |
| Sex | −1.59 ± 0.66 | 0.0157 |
| Mixed AD | | |
| Microvascular pathology (TLV) | 0.66 ± 0.65 | 0.3130 |
| Brain fraction (BF) | −2.75 ± 0.54 | <0.0001 |
| Age | −1.54 ± 0.63 | 0.0160 |
| Sex | −1.29 ± 1.13 | 0.2550 |

TABLE 6
The association between ADAS-cog₁₁ score and microvascular
pathology, brain atrophy, age, and sex in both AD subtypes.

| | Microvascular | Brain | Age | Sex | Overall |
|---|---|---|---|---|---|
| | pathology | atrophy | partial | partial | model |
| | partial R² | partial R² | R² | R² | R² |
| Typical AD | 4.18% | 9.96% | 1.11% | 1.21% | 15.86% |
| pattern | | | | | |
| Mixed AD | 1.35% | 9.16% | 2.54% | 0.57% | 12.39% |
| pattern | | | | | |

TABLE 7
Comparison of microvascular pathology and brain fraction
in the presence or absence of hypertension.

| | Hypertension | | |
|---|---|---|---|
| | Present | Absent | p-value |
| Microvascular pathology (cm³) | 3.72 ± 0.147 | 2.77 ± 0.184 | <0.0001 |
| Brain fraction (cm³) | 0.65 ± 0.06 | 0.66 ± 0.06 | 0.02519 |

1. A method of therapeutic or prophylactic treatment of microvascular brain disease in a subject,
which method comprises orally administering to said subject a methylthioninium (MT)-containing compound,
wherein said administration provides a total daily dose of between 8 and 200 mg of MT to the subject per day, optionally split into 2 or more doses,
wherein the MT-containing compound is a salt of 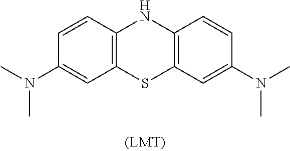
or a hydrate or solvate thereof.

2. A method as claimed in claim 1 wherein the treatment has a disease modifying effect on the microvascular pathology.

3. A method as claimed in claim 1 or claim 2 wherein the treatment reduces the rate of progression of microvascular pathology in the subject

4. A method as claimed in any one of claims 1 to 3 wherein the subject is a human.

5. A method as claimed in any one of claim 4 wherein the human subject is at least 60 years of age.

6. A method as claimed in any one of claims 1 to 5 wherein the subject has been diagnosed and/or selected as having said microvascular brain disease, optionally by brain imaging.

7. A method as claimed in any one of claims 1 to 5 wherein the subject has been assessed as being susceptible to, or at risk of, microvascular brain disease.

8. A method as claimed in claim 7 wherein the subject has been assessed as being susceptible or at risk based on familial or genetic data.

9. A method as claimed in claim 7 or claim 8 wherein the subject has been assessed as being susceptible or at risk based on age, history or presence of diabetes, hypertension.

10. A method as claimed in any one of claims 1 to 9 wherein the total daily dose is between 12 and 60 mg, or optionally between 16 and 32 mg.

11. A method as claimed in claim 10 wherein the total daily dose is about 16, 17, 18, 19, 20, 21, 22, 23, 24, 25, 26, 27, 28, 29, 30, 31, or 32 mg.

12. A method as claimed in any one of claims 1 to 11 wherein the total daily dose of the MT-containing compound is administered as a single dose or a split dose twice a day or three times a day.

13. A method as claimed in any one of claims 1 to 12 wherein the subject:
(i) has not historically received treatment with the neurotransmission modifying compound which is a modifier of the activity of acetylcholine or glutamate neurotransmitters, or
(ii) has historically received treatment with the neurotransmission modifying compound which is a modifier of the activity of acetylcholine or glutamate neurotransmitters, but ceased that treatment at least 1, 2, 3, 4, 5, 6, 7 days, or 2, 3, 4, 5, 6, 7, 8, weeks prior to treatment with the MT-containing compound, or
(iii) is selected as one who is receiving treatment with the neurotransmission modifying compound which is a modifier of the activity of acetylcholine or glutamate neurotransmitters, wherein said treatment is discontinued prior to treatment with the MT-containing compound.

14. A method as claimed in any one of claims 1 to 11 wherein the therapeutic treatment is not combined with a neurotransmission modifying compound which is a modifier of the activity of acetylcholine or glutamate neurotransmitters.

15. A method as claimed in any one of claims 13 to 14, wherein the neurotransmission modifying compound is an acetylcholinesterase inhibitor, optionally selected from donepezil; rivastigmine; and galantamine.

16. A method as claimed in any one of claims 13 to 14, wherein the neurotransmission modifying compound is an N-methyl-D-aspartate receptor (NMDA) receptor antagonist, which is optionally memantine.

17. A method as claimed in any one of claims 1 to 16 wherein the MT-containing compound is a compound of the following formula: 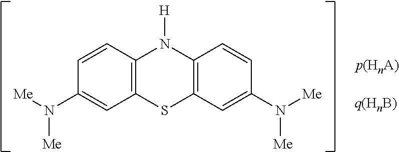
wherein each of HₙA and HₙB (where present) are protic acids which may be the same or different,
and wherein p=1 or 2; q=0 or 1; n=1 or 2; (p+q)×n=2.

18. A method as claimed in claim 17 wherein the MT-containing compound has the following formula, where HA and HB are different mono-protic acids:

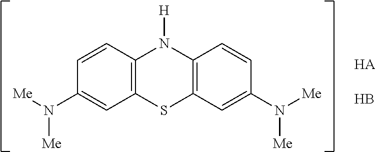

19. A method as claimed in claim 17 wherein the MT-containing compound has the following formula: 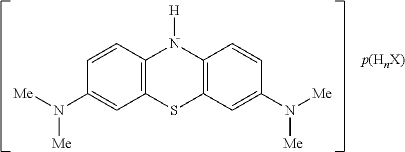
wherein each of HₙX is a protic acid.

20. A method as claimed in claim 17 wherein the MT-containing compound has the following formula and H₂A is a di-protic acid:

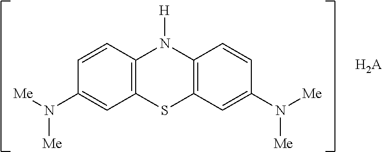

21. A method as claimed in claim 19 wherein the MT-containing compound has the following formula and is a bis-monoprotic acid:

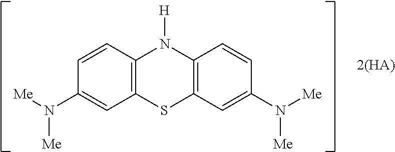

22. A method as claimed in any one of claims 17 to 21 wherein the or each protic acid is an inorganic acid.

23. A method as claimed in claim 22 wherein each protic acid is a hydrohalide acid.

24. A method as claimed in claim 22 wherein the or each protic acid is selected from HCl; HBr; HNO₃; H₂SO₄.

25. A method as claimed in any one of claims 17 to 21 wherein the or each protic acid is an organic acid.

26. A method as claimed in claim 25 wherein the or each protic acid is selected from H₂CO₃; CH₃COOH; methanesulfonic acid, 1,2-ethanedisulfonic acid, ethanesulfonic acid, naphthalenedisulfonic acid, p-toluenesulfonic acid.

27. A method as claimed in any one of claims 1 to 16, or claim 26 wherein the MT-containing compound is LMTM:

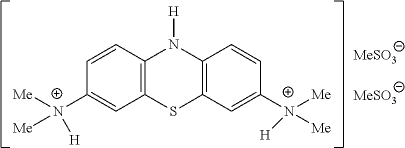

28. A method as claimed in any one of claims 1 to 42 wherein the MT-containing compound is selected from the list consisting of:

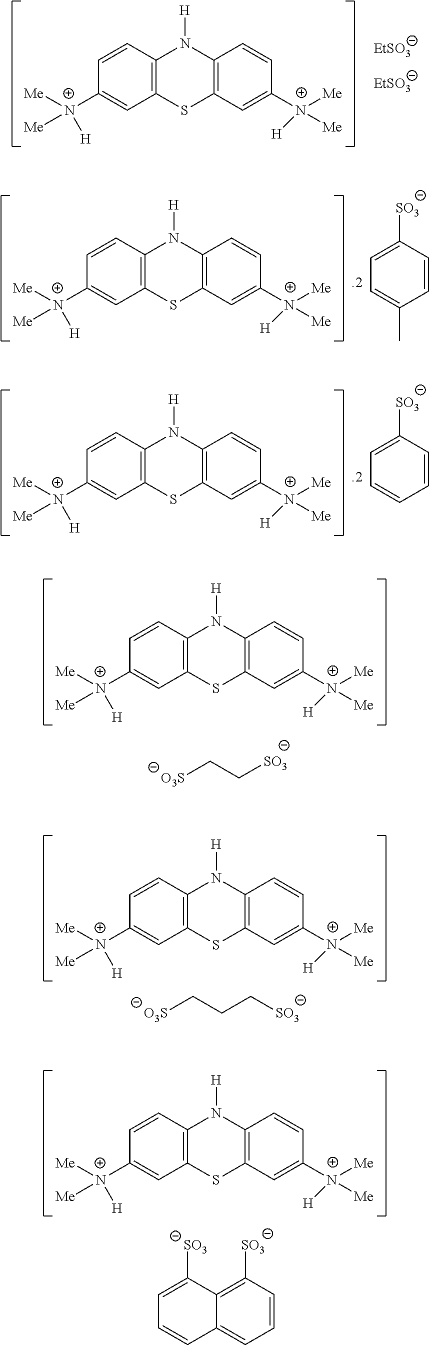

29. An MT-containing compound as defined in any one of claims 1 to 28, for use in a method of treatment as defined in any one of claims 1 to 28.

30. Use of an MT-containing compound or composition as defined in any one of claims 1 to 28, in the manufacture of a medicament for use in a method of treatment as defined in any one of claims 1 to 28.
